(19) United States
(12) Patent Application Publication
(10) Pub. No.: US 2004/0028677 A1
(43) Pub. Date: Feb. 12, 2004

(54) Chimeric allograft tolerance induction, monitoring and maintenance

(76) Inventor: Maria Z. Siemionow, Cleveland, OH (US)

Correspondence Address: Barbara E. Arndt,Ph.D., Jones Day, North Point, 901 Lakeside Avenue, Cleveland, OH, 44114, US

(21) Appl. No.: 10/443,281

(22) Filed: May 22, 2003

Related U.S. Application Data
(60) Provisional application No. 60/382,680, filed on May 22, 2002.

(51) Int. Cl. A61K 39/395; A61K 45/00

(52) U.S. Cl. 424/131.1; 424/93.7

(57) ABSTRACT

A method is provided for inducing donor-specific tolerance and/or mixed donor-recipient chimerism in an allograft transplant recipient, by administering to a recipient of an allograft a therapeutically effective amount of an immunosuppressive agent that depletes T cells; administering to the recipient of the allograft a therapeutically effective amount of anti-αβ T cell receptor antibodies; implanting an allograft from an allograft donor into the recipient; and implanting a therapeutically effective amount of bone marrow cells from the allograft donor into the allograft recipient.

This application claims the benefit of U.S. Provisional Application Serial No. 60/382,680, filed May 22, 2002, the entire disclosure of which is hereby incorporated by reference.

### BACKGROUND OF THE INVENTION

Recent advances in the field of clinical organ transplantation have made procedures such as heart, kidney and liver transplantation widely available to patients with end-organ disease. Various immunosuppressive drug regimens designed to control acute and chronic graft rejection play a vital role in facilitating modern transplantation. However, the requirement for life-long immunosuppressive drug therapy to prevent chronic graft rejection carries a significant risk of infectious and neoplastic complications due to the nonspecific immunosuppressive effects of these drugs.

The high morbidity and mortality associated with the chronic use of immunosuppressive agents has been a prohibiting factor in transplantation of composite tissue allografts (CTAs), such as hand, knee joint, larynx, and the like, leading to the search for new therapeutic strategies that would be more specific and less toxic.

In contrast to solid organ allografts, CTAs are neurovascularized allografts of tissues that include structural, functional, and aesthetic units of integumentary and musculoskeletal elements. Although not vital to life, CTAs are important to those who deal with the functional restoration of musculoskeletal defects. CTAs are unique in that they are a heterogeneous histological milieu of tissue elements, with each component possessing different antigen expression and presentation mechanisms. For example, CTAs can be comprised of several tissue types including skin, subcutaneous tissue, nerve and vascular tissues, bone, muscle, fascia, cartilage, and the like. In addition, CTAs can contain immunocompetent elements, such as bone marrow and lymph nodes, that can hasten the graft rejection processes and/or result in graft versus host disease (GVHD). The heterogeneous nature of CTAs not only affects the immune reactivity of these allogeneic tissues, but also defines potential immunomodulating strategies that may be different from those currently used in solid organ transplantation. That the majority of attempts at CTA transplantation have been unsuccessful illustrates the difficult barrier associated with a neurovascularized allograft comprised of a variety of tissues.

Several experimental designs to introduce new therapeutic strategies in transplantation have been reported. For example, one strategy is based on creation of stable hematopoietic chimerism within the transplant recipient. This approach requires preconditioning of the recipient to create a “space” needed for engraftment of the transplanted syngeneic and allogeneic bone marrow cells, by whole body irradiation, or other cytoablation procedures, and the like. However, the potential morbidity associated with preconditioning treatment regimens present a significant obstacle to the introduction of these strategies into use in clinical transplantation.

Therefore, it is a goal of allogeneic transplantation to provide effective, non-toxic treatments that can ensure indefinite graft survival. In particular, there is a need for the reliable induction and maintenance in the recipient of immunological tolerance to donor-specific antigens without the need for chronic immunosuppressive regimens or recipient preconditioning.

### SUMMARY OF THE INVENTION

The present invention provides a new approach for inducing long-term, donor-specific tolerance to donor antigens, especially in recipients of CTA transplants although not limited thereto, without the requirement for recipient preconditioning, without the need for chronic immunosuppressive regimens, and without the occurrence of GVHD. The methods according to the invention are fully applicable to transplantation of any type of allograft including, but not limited to, composite tissue such as, but not limited to human hand, human finger, human larynx, joints such as knee, hip, and the like; solid organs and glands such as, but not limited to, heart, lung, kidney, liver, pancreas, thyroid, and the like; glandular cells such as, but not limited to, islet cells and the like; skin; hematopoietic tissue; lymphoid tissue; tendons; ligaments; muscles; nerve tissue vascular tissue such as vessels; and the like, without limitation.

In one embodiment of the invention, a method is provided for inducing donor-specific tolerance and/or mixed donor-recipient chimerism in an allograft transplant recipient, comprising administering to a recipient of an allograft a therapeutically effective amount of an immunosuppressive agent that depletes T cells; administering to the recipient of the allograft a therapeutically effective amount of anti-αβ T cell receptor antibodies; implanting an allograft from an allograft donor into the recipient; and implanting a therapeutically effective amount of bone marrow cells from the allograft donor into the allograft recipient.

As used herein, mixed donor-recipient chimerism is used to described a state in which tissue or cells from a donor are able to live and function within a recipient host without rejection or the occurrence of GVHD. In a semi-allogeneic transplantation, where the donor and the recipient share at least one major histocompatibility complex (MHC) class I or class II locus, and the chimeric cells exhibit cell surface histocompatibility antigens of both the donor and the recipient (i.e., they are double positive). In a fully allogeneic transplantation, the donor and recipient do not share an MHC locus. In these chimeras, cells from the donor and cells from the recipient co-exist in the recipient, and these are both recognized as “self” and not rejected.

For purposes of this disclosure, the term “chimera” or “chimerism” is further intended to encompass trimeric and multimeric states such as, but not limited to, (a) states in which the recipient may have cells exhibiting both donor and recipient surface histocompatibility antigens, as well as cells from a third or multiple additional donors that are recognized as “self” by the recipient, all co-existing in the recipient; (b) states in which the recipient may have cells from three or multiple donors that are recognized as “self” by the chimeric recipient; and (c) all possible combinations and permutations of the foregoing, without limitation.

The immunosuppressive agent employed in the embodiments of the invention is preferably an inhibitor of the calcineurin pathway of T cell activation such as, but not limited to, cyclosporine A (CsA), FK-506, and the like; and/or other inhibitors of IL-2 production such as, but not limited to, rapamycin and the like, and combinations of the foregoing. More preferably, the immunosuppressive agent is CsA.

In a preferred embodiment, the immunosuppressive agent and the anti-αβ T cell receptor antibodies are administered as a short course of therapy that can be initiated prior to transplantation, alternatively at transplantation, alternatively about one to about three days after transplantation and, preferably, the therapy continues for a short time period after transplantation. In alternative embodiments, the immunosuppressive agent and the anti-αβ T cell receptor antibodies can be administered independently on a daily and/or non-daily basis during the treatment period of time, depending on the type of transplant, the type of donor, the condition of the recipient, and other factors, according to the judgement of the practitioner as a routine practice, without departing from the scope of the invention.

The donor bone marrow cells are preferably administered at the time of transplantation, but can be administered at any time up to about three days after transplantation. The embodiments of the invention also encompass readministering of donor bone marrow cells if the level of chimerism in the recipient declines and/or at the onset of allograft rejection.

The therapeutically effective amount of allograft donor bone marrow cells is preferably an amount sufficient to induce the production of mixed donor-recipient chimeric cells in the allograft recipient and, preferably, is an amount sufficient to maintain long-term recipient tolerance of the allograft without the necessity of readministration of the immunosuppressive agent and the anti-αβ T cell receptor antibodies. By long term tolerance is meant a period of time greater than 100 days, preferably greater than 300 days, more preferably greater than 720 days and, most preferably, lifelong survival of the allograft after cessation of the treatments.

In another embodiment, a combination of the steps of administering the immunosuppressive agent and the anti-αβ T cell receptor antibodies, and implanting of the donor bone marrow cells, results in induction of hematopoietic mixed donor-recipient chimerism and/or long-term allograft tolerance in the recipient.

In some embodiments, methods of implanting the donor bone marrow cells into the recipient include, but are not limited to, implantation and vascularization of a donor bone containing bone marrow, implantation of crude donor bone marrow, implantation of a suspension of nucleated donor bone marrow cells, implantation of a suspension of a nucleated cell subpopulation isolated from donor bone marrow cells, and the like. The term “crude” donor bone marrow is intended to encompass bone marrow that is harvested from a bone and has not necessarily undergone further processing. Preferably, the crude donor bone marrow is unprocessed marrow. Therefore, the crude bone marrow can include natural microanatomical components of the bone marrow including pluripotent stem cells, progenitor cells (including early progenitor cells), extracellular matrix, stromal elements, and the like, normally present in bone marrow. The term “suspension” of bone marrow cells is intended to encompass cells fluidly flushed from the bone marrow cavity and/or a suspension of cells obtained from processed crude bone marrow.

In other embodiments, isolated subpopulations of the donor bone marrow nucleated cells such as, but not limited to, tolerance inducing cells, hematopoietic stem cells, hematopoietic progenitor cells, CD4⁺ cells, CD8⁺ cells, and combinations of the foregoing, are implanted into the recipient. Such subpopulations can be isolated by methods well known in the art. In a further embodiment, a method is provided for inducing donor-specific tolerance and/or mixed donor-recipient trimerism in an allograft transplant recipient, comprising the steps of: (a) administering to a recipient of an allograft a therapeutically effective amount of an immunosuppressive agent that depletes T cells; (b) administering to the recipient of the allograft a therapeutically effective amount of anti-αβ T cell receptor antibodies; (c) implanting a first allograft from a first allogeneic allograft donor into the recipient; (d) implanting a therapeutically effective amount of bone marrow cells from the first allograft donor into the recipient; (e) implanting a second allograft from a second allogeneic allograft donor into the recipient; and (e) implanting a therapeutically effective amount of bone marrow cells from the second allograft donor into the allograft recipient. In still a further embodiment, the method can optionally include the steps of implanting additional allografts and bone marrow cells from additional donors, to induce tolerance to multiple donor allografts and/or mixed donor-recipient multimerism in the transplant recipient.

In further embodiments of the invention, methods are provided for monitoring allograft rejection in an allograft transplant recipient by implanting an additional allograft from the donor into the recipient. Preferably, the additional allograft can exhibit visible or histological signs that are readily ascertainable, and can indicate early recipient allograft rejection. As a non-limiting example, the additional allograft can include vascularized and/or non-vascularized skin, and the like. For example, a donor bone for implantation and vascularization in the recipient can include an attached portion of donor skin that also can be implanted and vascularized into the recipient as a monitor of allograft rejection. The additional, monitoring allograft can be especially useful as an indicator for monitoring graft rejection when the transplant recipient has received an internal allograft such as, but not limited to, a solid organ, or the like.

In yet other embodiments of the inventions, methods are provided for maintaining a level of mixed donor-recipient chimerism in an allograft transplant recipient. In one of these embodiments, the method comprises inducing donor-specific tolerance and/or mixed donor-recipient chimerism in an allograft recipient by any embodiment of the above-described methods, and maintaining a desired chimerism level by determining an optimal level of chimeric cells in the recipient not undergoing rejection of the allograft; harvesting chimeric cells from the recipient when an optimal level of chimeric cells is achieved; reconstituting the recipient with the harvested chimeric cells when the level of chimeric cells falls below a minimum level of chimeric cells; and, optionally, readministering an effective amount of the immunosuppressive agent and/or the anti-αβ T cell receptor antibodies sufficient to restore the desired chimerism level.

Embodiments of the invention include a system for inducing donor-specific tolerance and/or mixed donor-recipient chimerism in an allograft transplant recipient, comprising (a) a combination of pharmaceutical compositions for depletion of T cells in a recipient of an allograft from a donor, comprising an effective amount of a pharmaceutical composition that comprises an immunosuppressive T cell-depleting agent, and an effective amount of a pharmaceutical composition that comprises anti-αβ TCR⁺ T cell receptor antibodies, wherein administration of the combination to the recipient results in elimination of about 50% to about 99.9% of T cells circulating in peripheral blood of the recipient; and (b) a delivery system for implanting a therapeutically effective amount of bone marrow cells from the allograft donor into the allograft recipient, wherein the delivery system is selected from the group consisting of an implantable vascularizable bone from the allograft including the donor bone marrow; implantable crude donor bone marrow; an implantable donor bone marrow cell suspension; an implantable isolated subpopulation of nucleated donor bone marrow cells; and combinations of the foregoing.

The embodiments of the system and methods according to the invention are fully clinically applicable to transplantation in human recipients and, for example, are adaptable to take into account such uncertainties as the timing of the availability of allograft transplants for human recipients, and the like. Embodiments according to the invention are applicable to semi-allogeneic transplants such as, but not limited to, transplantation between related donor/recipients that are partially-mismatched at a major histocompatibility complex (MHC) class I or class II locus, and to fully-allogeneic transplants such as, but not limited to, transplantation between unrelated, fully mismatched MHC donor/recipient, including xenogeneic transplants to humans.

In the embodiments of the invention, the donor can be a mammal of a first species and the recipient can be a mammal of a second species. In further embodiments, the donor and the recipient can be of the same species. In yet further embodiments, the recipient is a primate. In a preferred embodiment, the recipient is a human. In embodiments employing human recipients, the anti-αβ TCR receptor antibodies preferably comprise human antibodies to human αβ TCR⁺ T cells. The anti-αβ TCR receptor antibodies are preferably monoclonal antibodies which can be humanized antibodies, but are preferably fully human polyclonal or monoclonal antibodies to human anti-αβ TCR receptors.

### BRIEF DESCRIPTION OF THE DRAWINGS

FIG. 1 illustrates flow cytometric (FC) evaluation of the peripheral blood αβ TCR+T cells in recipients receiving skin and crude bone marrow semi-allogeneic transplants and treated with combined CsA/αβ TCR mAb immunosuppressive therapy. FC analysis at day 7 post-transplantation demonstrated >90% depletion of the αβ TCR⁺ T cells, with gradual reconstitution to pre-transplant levels at day 63 post-transplantation. Sentinel (untransplanted) animals treated with the combined CsA/αβ TCR mAb served as controls.

FIG. 2 illustrates FC determination of the donor-originated RT-1ⁿ expression on CD4⁺ (A) and CD8⁺ (B) T cell subpopulations isolated from peripheral blood of recipients of skin and crude bone marrow transplantation and treated with combined CsA/αβ TCR mAb immunosuppressive therapy. Dot-plot results of the lymphoid cell subpopulations obtained from quadrangle analytical gates at day 65 after allotransplantation demonstrated the presence of the multilineage donor-specific chimerism ranging from 18.6% to 22.1% (RT-1ⁿ positive cells). Examination of two-color stained RT-1^{n-FITC}/CD4^{-PE} and RT-1^{n-FITC}/CD8^{-PE} peripheral lymphocytes revealed 4.1% and 7.4%, respectively, of double positive CD4 and CD8 T cell subpopulations.

FIG. 3 illustrates FC analysis of the CD90⁺antigen expression on the surface of stem and progenitor cells from donor bone marrow before and after selection using a magnetic MiniMACS separating system. FIG. 3A shows CD90⁺ cells as the small peak, and the remainder of the bone marrow nucleated cells as the larger peak, prior to selective separation of the CD90⁺ cells from a suspension of bone marrow cells. The CD90⁺ cells comprised about 22% of the bone marrow nucleated cells. FIG. 3B shows the purity of separation of the CD90⁺ cells (large peak). Less than 5% were CD90⁻. Over 95% of analyzed cells expressed the CD90 antigen, indicating high efficacy of the selection.

FIG. 4 illustrates intraosseous transplantation of the donor-derived stem and progenitor cells. FIG. 4A illustrates the injection of the stem and progenitor cells directly into the bone marrow cavity of the recipient's left tibia. Following injection, the right hindlimb from the same donor was transplanted to the recipient (FIG. 4B).

FIG. 5 illustrates a hindlimb allograft survival chart indicating significant extension of hindlimb allograft survival (p<0.05) following perioperative injection of 8-12×10⁵ stem and progenitor cells (CD90⁺ cells) directly into the bone marrow cavity of the hindlimb allograft recipients without an immunosuppressive protocol.

FIG. 6 illustrates a two-color flow cytometric analysis at day 14 after hindlimb allograft transplantation, showing transient chimerism in the allograft control treatment (0.6%, A) and a high level (3.4%, B) of double positive RT-1¹⁺ⁿ/CD4⁺ chimeric cells in the peripheral blood of limb recipients treated with the intraosseous injection of donor CD90⁺ stem and progenitor cells at the time of transplantation.

FIG. 7 illustrates flow cytometric analysis at the day 35 after hindlimb allograft transplantation, showing high levels of multilineage donor-specific lymphoid chimerism (B1-B3) in the peripheral blood mononuclear cells (PMBC) of the recipients receiving direct intraosseous injection of donor stem and progenitor cell. In contrast, intravenous injection of the same number of cells resulted in low-level, transient chimerism (A1-A3), indicating that bone creates more permissive conditions for donor stem and progenitor cell engraftment.

FIG. 8 illustrates vascularized skin and bone allografts (VSBA). A schematic representation of the VSBA model combining a superficial epigastric skin flap and a vascularized femoral bone allograft (A). B shows a Giemsa stained vascularized bone marrow isograft 7 days after transplantation, showing over 99% viability of the bone marrow cells in these transplants. C shows an accepted VSBA transplant allograft across a fully mismatched major MHC barrier (Brown Norway donor, Lewis recipient) at day 63 after cessation of immunosuppressive protocol, showing full acceptance of the vascularized skin allograft. D shows the skin biopsy (hematoxylin and eosin stained) with preserved dermis and epidermis and no histological signs of rejection. E shows the immunohistostained frozen sections of the bone marrow isolated from the donor vascularized bone allograft after transplantation into the recipient, at day 63 after cessation of immunosuppressive protocol. F illustrates flow cytometry analysis of isolated cells. More than 50% of the cells in the donor bone marrow were recipient CD90⁺/RT-1^{L} (related to LEW MHC class I) positive cells, showing the trafficking of recipient cells into the donor bone marrow, and also confirming the viability of the transplanted vascularized bone marrow.

FIG. 9 illustrates a LEW recipient of two genetically unrelated VSBA transplants at day 35 after transplantation. The vascularized bone transplants are not visible, as they are beneath the transplanted skin flaps shown. On the left is a VSBA transplant from a BN donor, showing full skin acceptance by the LEW recipient of this fully allogeneic transplant. On the right is a VSBA transplant from an ACI donor, showing full skin acceptance by the LEW recipient of this fully allogeneic transplant.

FIGS. 10A and 10B illustrate H&E stained formalin-fixed skin tissues taken from the BN allograft and the ACI allograft, respectively, at day 21 after transplantation, showing preserved dermis and epidermis and no histological signs of rejection.

FIG. 11 illustrates flow cytometry analysis performed on PBMC of the LEW recipients at day 21 after transplantation of VSBA transplants from both the BN and the ACI donors. The dot-plot results of the lymphoid cell subpopulations obtained from quadrangle analytical gates demonstrated the presence of double positive RT-1^{a-FITC}/CD4^{-PE, RT-}1^{a-FITC}/CD8^{-PE} and RT-1^{a-FITC}/CD45RA^{-PE} (ACI/LEW) at a level of 8.02%, 4.36% and 0.82%, respectively, of PBMC (top horizontal row); and also the presence of double positive RT-1^{n-Cy7}/CD4^{-PE}, RT-1^{n-Cy7}/CD8^{-PE} and RT-1^{n-Cy7}/CD45RA^{-PE} (BN/LEW) at a level of 0.9%, 0.3% and 4.1%, respectively (bottom horizontal row).

FIG. 12 illustrates flow cytometry analysis performed on PBMC of the LEW recipients at day 35 after transplantation of VSBA transplants from both the BN and the ACI donors. The dot plot results demonstrated the presence of double positive RT-1^{a-FITC}/CD4^{-PE}, RT-1^{a-FITC}/CD8^{-PE} and RT-1^{a-FITC}/CD45RA^{-PE} (ACI/LEW) at a level of 7.99%, 4.73% and 0.6%, respectively, of PBMC (top horizontal row); and also the presence of double positive RT-1^{n-Cy7}/CD4^{-PE}, RT-1^{n-Cy7}/CD8^{-PE} and RT-1^{n-Cy7}/CD45RA^{-PE} (BN/LEW) at a level of 0.8%, 0.48% and 3.1%, respectively (bottom horizontal row).

### DETAILED DESCRIPTION OF THE INVENTION

The present invention provides methods for achieving long-term allograft survival without chronic immunosuppression and without the requirement for recipient preconditioning, without the need for chronic immunosuppressive therapy, and without the occurrence of GVHD. The methods described and claimed herein are especially useful for human patients requiring transplantation of non-vital organs, including, but not limited to, those needing skin replacement after devastating burn injuries, cancer patients who need customized replacement of large parts of their bodies, immobilized rheumatoid patients requiring replacement of several joints, children born with congenital defects, and the like. Moreover, embodiments of the methods of the invention can be useful for solid organ and gland transplants, and allografts for treatment of inborn errors of metabolism, leukemias, immunodeficiency syndromes, GVHD relapse, and the like, without limitation.

In one embodiment according to the invention, a method is provided for inducing donor-specific tolerance and/or mixed donor-recipient chimerism in an allograft transplant recipient, comprising the steps of: (a) administering to a recipient of an allograft a therapeutically effective amount of an immunosuppressive agent that depletes T cells; (b) administering to the recipient of the allograft a therapeutically effective amount of anti-αβ T cell receptor antibodies; (c) implanting an allograft from an allograft donor into the recipient; and (d) implanting a therapeutically effective amount of bone marrow cells from the allograft donor into the allograft recipient.

Preferably, the immunosuppressive agent and the anti-αβ T cell receptor antibodies are administered according to a protocol such as those disclosed and claimed in our co-owned, copending U.S. patent application Ser. No. 10/427,013, filed Apr. 30, 2003, entitled, “Induction and Maintenance of Tolerance to Composite Tissue Allografts,” the entire disclosure of which is hereby incorporated by reference.

The immunosuppressive agent and the anti-αβ T cell receptor antibodies are preferably administered in an amount, at a frequency, and for a duration of time sufficient to induce donor-specific tolerance and/or mixed donor-recipient chimerism in the allograft recipient.

An immunosuppressive agent, as used herein, is an agent such as a chemical agent or a drug that, when administered at an appropriate dosage over an appropriate time period, results in the depletion of T cells, preferably mature T cells. The immunosuppressive agent is preferably an inhibitor of the calcineurin pathway of T cell activation such as, but not limited to, cyclosporine A (CsA), FK-506, and the like; or other inhibitors of IL-2 production such as, but not limited to, rapamycin and the like, and combinations of the foregoing. More preferably, the immunosuppressive agent is CsA.

The above-identified patent application, incorporated by reference herein, discloses and claims methods for achieving long-term CTA survival without chronic immunosuppression or recipient preconditioning by employing a new approach based on the pivotal role of T cells in the rejection of allografts. Briefly, T cell recognition of foreign major histocompatibility complex (MHC) antigens plays a crucial role in the initiation of allograft rejection. T lymphocytes are classified as αβ or γδ depending on the type of disulfide-linked heterodimeric glycoprotein T cell receptor (TCR) displayed. The T cells responsible for most immune responses, including allograft rejection, are the T cells bearing αβ T cell receptors (αβ TCR⁺ T cells). It was discovered that anti-αβ TCR antibodies, preferably monoclonal (mAb) anti-αβ TCR antibodies, can successfully be employed to specifically eliminate αβ TCR⁺ T cells to create a window of immunological incompetence in the transplant recipient. While not being bound by theory, it is believed that subsequent repopulation of the recipient thymus by αβ TCR⁺ T cells, in the presence of donor alloantigens, results in the induction of hematopoietic mixed donor-recipient chimerism in the transplant recipient and the long-term immunological tolerance demonstrated in these recipients.

It had previously been demonstrated that treatment with the anti-αβ TCR mAb alone reduced T cell numbers in a dose-dependent manner, with a significant reduction after one dose of antibody; however T cell depletion did not progress further with continued antibody therapy. While not being bound by theory, it is believed that some T cells in the blood, lymphatic vessels or lymphatic organs, such as the spleen, lymph nodes thymus, or the like, may have escaped initial exposure to the depleting antibody and repopulated rapidly to reject the graft. To address this apparent “T cell escape phenomenon,” an immunosuppressive agent, in addition to the anti-αβ TCR mAb, was employed to prevent the rejection response by reducing allograft-responsive T cell proliferation and enhancing the effectiveness of the depletion protocol.

Although it is considered virtually not possible to eliminate all αβ TCR⁺ cells by the combined immunosuppressive and antibody therapy, it is preferable that the combined treatment be effective to eliminate about 50% to about 99.9%, preferably about 75% to about 95%, more preferably about 80% to at least about 90% of the αβ TCR⁺ cells during the short course of therapy. The embodiments of the method of the invention provide significant depletion of the recipient T-cell population at the end of the immunodepleting therapy, as well as allow repopulation of the recipient T cell repertoire once the treatment protocol is withdrawn. In another embodiment, the immunosuppressive T cell deleting agent and the anti-αβ TCR⁺ T cell receptor antibodies are administered as a pharmaceutical composition that comprises both the immunosuppressive agent and the antibodies.

The anti-αβ T cell receptor antibodies employed in embodiments of the methods according to the invention are preferably monoclonal (mAb) αβ T cell receptor antibodies, and are generally commercially available or can be produced by known methods without undue experimentation. Non-monoclonal anti-αβ T cell receptor antibodies with suitable specificity and an efficacy similar to monoclonal αβ T cell receptor antibodies, or whose epitope overlaps that of the monoclonal antibody, are also suitable. It is also known that hybridomas producing monoclonal antibodies may be subject to genetic mutation or other changes while still retaining the ability to produce monoclonal antibody of the same desired specificity. The embodiments of the invention methods therefore encompass mutants, other derivatives and descendants of the hybridomas producing anti-αβ TCR mAbs. It is also known that a monoclonal antibody can be subjected to the techniques of recombinant DNA technology to produce other derivative antibodies, humanized or chimeric molecules or antibody fragments that retain the specificity of the original monoclonal antibody. Such techniques may involve combining DNA encoding the immunoglobulin variable region, or the complementarity determining regions (CDRs) of the monoclonal antibody with DNA coding the constant regions, or the constant regions plus framework regions, of a different immunoglobulin, for example, to convert a mouse-derived monoclonal antibody into one having largely human immunoglobulin characteristics. (See, for example, EP 184187A and GB 2188638A.) The embodiments of the invention also encompass humanized monoclonal antibodies to the αβ TCR epitopes. In a most preferred embodiment, fully human antibodies to human αβ TCR epitopes are employed in human recipients. These human antibodies can be polyclonal with suitable specificity and efficacy and, preferably, are human monoclonal antibodies.

As disclosed in the above application incorporated by reference, a significant depletion of the T cell population was demonstrated at the end of immunodepleting regimens that included a combination of CsA and anti-αβ TCR antibodies, as well as repopulation of the recipient T cell repertoire once the treatment protocol had been withdrawn. None of the immunodepleted recipients showed signs of graft versus host disease (GVHD) or other signs of recipient immunodeficiency. It was also demonstrated that introduction of a 5-day, 7-day, or up to a 35-day short-term immunosuppressive protocol of combined CsA and anti-αβ TCR antibodies that were initially administered up to about 24 hours prior to transplantation in semi-allogeneic recipients of composite tissue allografts, resulted in induction and maintenance of donor-specific tolerance across a major histocompatibility barrier without the need for longer term, chronic immunosuppression. This was confirmed by the indefinite (>750 days) survival of the allografts in the recipients treated with the combined therapy. Using a protocol in which the combined CsA and anti-αβ TCR antibodies were initially administered at about the time of transplantation and daily for 5 days or 7 days only, tolerance was induced in fully-MHC mismatched allogaft transplants without the occurrence of GVHD, and donor specific tolerance was confirmed by the mixed lymphocyte reaction (MLR). The fully-allogeneic recipients have lived over 450 days after cessation of therapy, and continue to live.

Regardless of the immunosuppressive protocols employed, all of the recipients of the combined CsA/αβ-TCR mAb treatment were immunocompetent, as verified by a mixed lymphocyte reaction and the observation that the treated, allotransplanted recipients uniformly rejected third part grafts. Moreover, the immunocompetence of these recipients was verified biologically, as none of the animals over the entire range of protocols (including all recipients of semi-allogeneic and fully-allogeneic transplants and the combined treatment protocols) showed signs of disease, including viral infection or lymphoma formation, over 720 days post-transplant (which is a typical life span for a rat).

Mixed hematopoietic donor-recipient chimeric cells were identified in the peripheral blood of the semi-allogeneic and fully allogeneic allograft recipients by flow cytometry using our novel three-color immunostaining technique. In addition to CD4⁺ and CD8⁺ donor-recipient chimeric cells, CD90⁺ stem and progenitor cells and/or CD45RA⁺ B cell populations and/or CD90⁺/CD45RA⁺ B cell progenitor populations were found to be permissive cell populations facilitating tolerance induction (tolerance inducing cells, TICs). Without being bound by theory, it is believed that, since the vascularized bone marrow is an integral part of limb allografts, stem cells and progenitor cells of donor bone marrow origin become engrafted in the recipient lymphoid organs during immunosuppressive therapy according to embodiments of the invention, resulting in induction of hematopoietic mixed donor-recipient chimerism in the recipient. Circulating chimeric cells can be identified in the peripheral blood and/or lymphoid organs of the recipients by staining with a monoclonal antibody specific for a donor peripheral blood mononuclear cell (PBMC) antigen. A low level of hematopoietic chimerism as a mechanism of tolerance induction is supported by other studies in which we found that rats rejected uniformly skin flap allografts devoid of bone marrow despite the CsA/αβ-TCR mAb treatment. In contrast, skin grafts transplanted simultaneously with bone marrow of donor origin were accepted (over 80 days) across an MHC barrier.

In embodiments of the present invention, administration of the therapeutically effective amount of a combination of anti-αβ T cell receptor antibodies and the immunosuppressive agent capable of depleting T cells, preferably mature T cells, can be given prophylactically or therapeutically. By “prophylactic,” it is meant the protection, in whole or in part, against allograft rejection. By “therapeutic,” it is meant the amelioration of allograft rejection itself, and the protection, in whole or in part, against further allograft rejection. The antibodies and immunosuppressive drugs, as used herein, include all biochemical equivalents thereof (i.e., salts, precursors, the basic form, and the like).

In a preferred embodiment, the immunosuppressive agent and the anti-αβ T cell receptor antibodies are administered as a short course of therapy that can be initiated prior to transplantation, alternatively at transplantation, alternatively about one to about three days after transplantation and, preferably, continues for a short time period after transplantation. In an embodiment of the invention that is particularly useful for semi-allogeneic transplantation, the immunosuppressive agent and the anti-αβ T cell receptor antibodies can be initially administered at about the time of transplantation to about 24 hours prior to transplantation, preferably about 12 hours to about 24 hours prior to transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation.

In another embodiment of the invention that is particularly useful for fully-allogeneic transplantation, the immunosuppressive agent and the anti-αβ T cell receptor antibodies are initially administered during a period of time from about one hour prior to transplantation to at the time of transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation. For fully allogeneic transplantation, it is more preferable initially to administer the immunosuppressive agent and the anti-αβ T cell receptor antibodies at about the time of transplantation, in order to avoid the occurrence of GVHD in these recipients.

In another embodiment, the immunosuppressive agent and the anti-αβ T cell receptor antibodies are first administered from one to three days after transplantation, and daily administration continues for a period of time of about 100 days, about 50 days, about 35 days, about 21 days, about 14 days, preferably for about 7 days or, especially, for about 5 days after transplantation.

In alternative embodiments, the immunosuppressive agent and the anti-αβ T cell receptor antibodies can be administered independently on a daily and/or non-daily basis during the treatment period of time, depending on the type of transplant, the type of donor, the condition of the recipient, and other factors, according to the judgement of the practitioner as a routine practice, without departing from the scope of the invention. In another embodiment, the immunosuppressive T cell deleting agent and the anti-αβ TCR⁺ T cell receptor antibodies are administered as a pharmaceutical composition that comprises both the agent and the antibodies.

The immunosuppressive agent(s) and/or the antibodies useful in embodiments of the invention can be a pharmaceutically acceptable analogue or prodrug thereof, or a pharmaceutically acceptable salt of the immunosuppressive agent(s) or antibodies disclosed herein, which are effective in inducing long-term, donor specific tolerance to allografts. By prodrug is meant one that can be converted to an active agent in or around the site to be treated.

Treatment will depend, in part, upon the particular therapeutic composition used, the amount of the therapeutic composition administered, the route of administration, and the cause and extent, if any, of the disease.

The antibodies and immunosuppressive agent(s) described herein, as well as their biological equivalents or pharmaceutically acceptable salts can be independently or in combination administered by any suitable route. The manner in which the agent is administered is dependent, in part, upon whether the treatment is prophylactic or therapeutic. Although more than one route can be used to administer a particular therapeutic composition, a particular route can provide a more immediate and more effective reaction than another route. Accordingly, the described routes of administration are merely exemplary and are in no way limiting. Suitable routes of administration can include, but are not limited to, oral, topical, subcutaneous and parenteral administration. Examples of parenteral administration include, but are not limited to, intravenous, intraarterial, intramuscular, intraperitoneal, and the like.

The dose of immunosuppressive agent, anti-αβ T cell receptor antibodies, and/or donor bone marrow cells administered to an animal, particularly a human, in accordance with embodiments of the invention, should be sufficient to effect the desired response in the animal over a reasonable time frame. It is known that the dosage of therapeutic agents depends upon a variety of factors, including the strength of the particular therapeutic composition employed, the age, species, condition or disease state, and the body weight of the animal. Moreover, the dose and dosage regimen will depend mainly on whether the compositions are being administered for therapeutic or prophylactic purposes, separately or as a mixture, the type of biological damage to the host, the type of host, the history of the host, and the type of immunosuppressive agents or biological active agent. The size of the dose will be determined by the route, timing and frequency of administration as well as the existence, nature and extent of any adverse side effects that might accompany the administration of a particular therapeutic composition and the desired physiological effect. It is also known that various conditions or disease states, in particular, chronic conditions or disease states, may require prolonged treatment involving multiple administrations. Therefore, the amount of the agent and/or antibodies must be effective to achieve an enhanced therapeutic index.

It is noted that humans are generally treated longer than mice and rats with a length proportional to the length of the disease process and drug effectiveness. The therapeutic purpose is achieved when the treated hosts exhibit improvement against disease or infection, including but not limited to improved survival rate of the graft and/or the host, more rapid recovery, or improvement in or elimination of symptoms. If multiple doses are employed, as preferred, the frequency of administration will depend, for example on the type of host and type of disease. The practitioner can ascertain upon routine experimentation which route of administration and frequency of administration are most effective in any particular case. Suitable doses and dosage regimens can be determined by conventionally known range-finding techniques. Generally, treatment is initiated with smaller dosages, which are less than the optimum dose of the compound. Thereafter, the dosage is increased by small increments until the optimum effect under the circumstances is reached.

The dose and dosage regimen will depend mainly on whether the compositions are being administered for therapeutic or prophylactic purposes, separately or as a mixture, the type of biological damage and host, the history of the host, and the type of immunosuppressive agent or biologically active agent. The amount must be effective to achieve an enhanced therapeutic index. It is noted that humans are generally treated longer than rats with a length proportional to the drug effectiveness. The doses may be single doses or multiple doses over a period of several days. Therapeutic purposes are achieved as defined herein when the treated hosts exhibit allograft tolerance, including but not limited to improved allograft survival rate, more rapid recovery, or improvement or elimination of transplantation-associated symptoms. If multiple doses are employed, as preferred, the frequency of administration will depend, for example, on the type of host and type of allograft, dosage amounts, and the like.

Compositions for use in the present inventive method preferably comprise a pharmaceutically acceptable carrier and an amount of the therapeutic composition sufficient to induce tolerance prophylactically or therapeutically. The carrier can be any of those conventionally used and is limited only by chemical-physical considerations, such as solubility and lack of reactivity with the compound, and by the route of administration. It will be appreciated by one of ordinary skill in the art that, in addition to the following described pharmaceutical compositions, the therapeutic composition can be formulated as polymeric compositions, inclusion complexes, such as cyclodextrin inclusion complexes, liposomes, microspheres, microcapsules and the like.

The therapeutic composition can be formulated as a pharmaceutically acceptable acid addition salt. Examples of pharmaceutically acceptable acid addition salts for use in the pharmaceutical composition include those derived from mineral acids such as, but not limited to, hydrochloric, hydrobromic, phosphoric, metaphosphoric, nitric and sulfuric acids, and the like, and organic acids such as, but not limited to, tartaric, acetic, citric, malic, lactic, fumaric, benzoic, glycolic, gluconic, succinic, and arylsulphonic, for example p-toluenesulphonic, acids, and the like.

The pharmaceutically acceptable excipients described herein, for example, vehicles, adjuvants, carriers or diluents, are well-known to those who are skilled in the art and are readily available to the public. It is preferred that the pharmaceutically acceptable carrier be one which is chemically inert to the therapeutic composition and one which has no detrimental side effects or toxicity under the conditions of use.

The choice of excipient will be determined in part by the particular therapeutic composition, as well as by the particular method used to administer the composition. Accordingly, there are a wide variety of suitable formulations of the pharmaceutical composition of the present invention. The formulations described herein are merely exemplary and are in no way limiting.

Injectable formulations are among those that are preferred in accordance with the present inventive method. The requirements for effective pharmaceutically carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)). It is preferred that such injectable compositions be administered intramuscularly, intravenously, or intraperitoneally.

Topical formulations are well-known to those of skill in the art. Such formulations are suitable in the context of the present invention for application to the skin in a form such as, but not limited to, patches, solutions, ointments, and the like.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compositions can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethyl-cellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metals, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof. The parenteral formulations will typically contain from about 0.5 to about 25% by weight of the active ingredient in solution. Preservatives and buffers may be used.

In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The present inventive method also can involve the co-administration of other pharmaceutically active compounds. By “co-administration” is meant administration before, concurrently with, e.g., in combination with anti-cancer composition in the same formulation or in separate formulations, or after administration of a therapeutic composition as described above. For example, corticosteroids, e.g., prednisone, methylprednisolone, dexamethasone, or triamcinalone acetinide, or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered.

In our studies using a rat hindlimb (CTA) allograft transplantation model, a remarkably stable hematopoietic donor-recipient chimerism was observed in the recipients as evidenced by a peripheral blood chimeric cell level of about 20% to about 30% of circulating mononuclear cells, and greater than about 60% chimeric cells in lymphoid tissue. Without being bound by theory, this finding suggests that the presence of certain tissue compartments in the composite tissue allografts may facilitate efficient engraftment of the donor hematopoietic cells and contribute to the observed long-term tolerance (over 750 days) across a major histocompatibility complex (MHC) barrier in a multitissue allograft. It is known that bone marrow stromal cells play a critical role in the formation of the hematopoietic microenvironment and support hematopoictic stem cell differentiation through the inter-cellular contact and secretion of various cytokines and growth factors. The hematopoietic microenvironment that is created by transplantation of marrow stromal cells, strains or crude bone marrow allows for the ectopic development of a hematopoietic tissue at the site of transplantation. Again, without being bound by theory, it is possible that microanatomic environment and stromal components in the bone component of the allograft can provide essential support elements that allow for the successful mismatched transplant without recipient preconditioning and contribute to the ability to induce a tolerant state allowing for stable chimerism.

We have now discovered that enhancement of CTA survival can be achieved, with or without the development of enhanced mixed donor-recipient chimerism, by the combination of immunosuppressive agent and the anti-αβ T cell receptor antibodies and an additional implantation of a therapeutically effective amount of bone marrow cells from the same donor that provided the allograft. Moreover, we have discovered that the enhanced allograft survival and mixed chimerism can be achieved by implantation of the donor bone marrow cells employing any delivery system including, but not limited to, an implantable, vascularizable bone from the allograft donor including the donor bone marrow; implantable crude donor bone marrow; an implantable donor bone marrow cell suspension; an implantable isolated subpopulation of nucleated donor bone marrow cells; and combinations thereof.

In one embodiment of the invention, the step of implanting bone marrow cells comprises the substeps of implanting and vascularizing an allograft donor bone including the bone marrow into the recipient. This method is intended to encompass the composite tissue allograft model described in the patent application incorporated herein by reference. However, preferably this method involves the implantation of the donor bone separately from another allograft such as, but not limited to, a solid organ or any of the aforementioned allografts listed above, without limitation. The implanted bone is most preferably vascularized by anastomosis with the recipient's vascular tissue, such that the bone has a blood flow provided by the recipient, thus allowing trafficking between the donor and recipient hematopoietic and circulatory compartments. Implantation of vascularized bone can be accomplished by any known surgical technique. For example, a non-limiting technique, such as that described in a rat model example below, can be adapted to human transplant recipients without undue experimentation. Briefly, vascularized femoral bone allograft can be harvested on the femoral artery and vein of the donor, preserving supplying collateral vessels. The bone allograft can be transferred to the recipient's groin region and end-to-end anastomoses between donor's and recipients femoral arteries and veins can be performed using standard microsurgical techniques. Other vascularizable bone allografts and other sites for vascularized bone allograft implantation can also be selected, without limitation.

In another embodiment of the invention, the allograft donor bone can further include an attached portion of donor skin, and the substeps of implanting and vascularizing the donor bone into the recipient can further include the substeps of implanting and vascularizing the attached portion of donor skin. This embodiment is especially useful for providing an easily accessible additional allograft for purposes of monitoring allograft rejection. Implantation of skin allografts is well known and implantation of skin vascularized by anastomosis with the recipient's vascular tissue can be accomplished by any known surgical technique. For example, a non-limiting technique described in a rat model exemplified below can be adapted to human transplant recipients without undue experimentation. Briefly, a standard template can be used to mark the flap borders both in the donor and the recipient. The donor skin flap can be elevated on the superficial epigastric branch of the femoral artery and vein of the donor, and end-to-end anastomoses can be performed between the donor's and recipient's femoral arteries and veins using standard microsurgical techniques. Other sources of vascularizable skin and other sites for vascularized skin implantation can also be selected, without limitation.

Skin grafts that are not vascularized can also be implanted for purposes of monitoring allograft rejection, by well known standard surgical techniques.

In another embodiment of the invention, a method is provided for crude donor bone marrow transplantation directly into the medullary cavity of the transplant recipient bone. This method allows avoidance of recipient preconditioning for creation of immunological silence, and preservation of the microenvironment for the bone marrow cells during transplantation. The method provides for direct engraftment of donor-derived bone marrow stem and progenitor cells and, as a result, the establishment of donor-specific hematopoietic chimerism. That is, the method provides preservation of the donor bone marrow microenvironment for creation of a chimeric state in the recipient without the need for recipient preconditioning (such as whole body irradiation or the like) or for vascularized bone marrow transplantation, such as by composite allograft transplants, vascularized bone implantation, or the like.

The direct transplantation of the donor bone marrow in the crude form into the medullary cavity of the long bones of transplant recipient allows not only for the engraftment of the bone marrow cells, but also provides a natural matrix as an ideal microenvironment for cell engraftment for subsequent cell repopulation and trafficking. As described in the examples below, the engraftment of the donor specific bone marrow cells and their trafficking into the periphery was confirmed by flow cytometry, revealing 4.1% and 7.4% of donor specific RT-1^{n-FITC}/CD4^{-PE} and RT-1^{n-FITC}/CD8^{-PE} chimeric cells, respectively, in the peripheral blood of recipients at day 63 after cessation of the immunosuppressive treatment protocol. The induction of this mixed, donor specific chimerism resulted a the significant extension of the skin allograft survival in recipients of the donor crude bone marrow. This method of crude bone marrow transplantation is simple, minimally invasive, does not require recipient preconditioning and can be easily implemented into clinical practice in humans.

The step of implanting donor bone marrow cells into the recipient by direct intraosseous transfer of donor crude bone marrow can comprise the substeps of obtaining crude bone marrow from a bone marrow cavity of the allograft donor, and implanting the donor crude bone marrow into the allograft recipient. Any suitable method can be employed to obtain the crude bone marrow such as, but not limited to, scooping the marrow from the donor bone and depositing the scooped marrow directly into the bone marrow cavity of a recipient bone. Optionally, it may be desirable to create a space in the recipient bone marrow cavity prior to receiving the donor bone marrow. As a non-limiting example, it may be desirable to remove a roughly equivalent amount of recipient marrow to that of the donor marrow to be received. However, neither creation of the space nor the amount of recipient bone marrow removed are critical to the present invention.

In another embodiment of obtaining crude donor bone marrow and implanting the same into a recipient bone marrow cavity, a pumping device may be employed. That is, an amount of recipient marrow can be withdrawn through one cannula, and an amount of donor marrow can be implanted through another cannula. These steps can be repeated as often as necessary to complete the delivery of a desired amount of the crude donor bone marrow. The pumping device can be any suitable device, including a two-way syringe mechanism, without limitation.

The amount of donor crude bone marrow to be delivered to the recipient will depend on numerous factors such as, but not limited to, the type and condition of the recipient, the route of administration, and the like as described above, and can be tailored to each recipient according to normal medical practice. A therapeutically effective amount of donor crude bone marrow implanted into the recipient is that sufficient to induce allograft tolerance and/or the production of mixed donor-recipient chimeric cells in the recipient.

In another embodiment of the invention, the step of implantation of the donor bone marrow cells comprises the substeps of obtaining a bone marrow cell suspension from a bone of the allograft donor, and implanting the bone marrow cell suspension into the recipient. The suspension of bone marrow cells can be prepared by fluidly flushing the bone marrow cells from the bone marrow cavity is intended to encompass cells fluidly flushed from the bone marrow cavity and/or a suspension of cells obtained by processing crude bone marrow. The former flushing method is preferred. In a non-limiting example, freshly isolated donor bone such as, but not limited to, the femur, tibia, and the like, are isolated and two contralateral ends of the bones can be cut and the residual bone marrow cells flushed out from the bone marrow cavity using a syringe-based pump system. After lysis of red blood cells, nucleated marrow cells are then washed and counted to obtain a final concentration of cells in a physiological medium, such as phosphate buffered saline, or the like.

The suspension of bone marrow cells is then employed for implantation into the allograft recipient by direct intraosseous transplantation into a recipient bone marrow cavity or by intravenous injection into the recipient. Alternatively, as described further below, implantation into the allograft recipient can be by direct intraosseous transplantation into an implanted vascularized donor bone or donor bony scaffold.

Isolated bone marrow cell subpopulations, such as pluripotent stem cells or progenitor cells can be obtained by further processing the foregoing bone marrow suspensions. Isolation methods for cell subpopulations are well known. For example, stem cells and progenitor cells can be separated on the basis of their differential staining with rhodamine; stem cells do not stain with rhodamine, whereas progenitor cells do stain with rhodamine. Exemplary suitable methods for isolation of cell subpopulations include, but are not limited to, separation of subpopulations by a fluorescence activated cell sorter, by positive and/or negative selection by magnetic beads coated with an appropriate antibody, and the like, without limitation. Assessment of the purity of the desired cell populations can be accomplished by flow cytometry, or other known methods.

After isolation of the bone marrow subpopulation, a desired number of cells in suspension can be implanted into the recipient by direct intraosseous, intravenous routes, or the like, as described for bone marrow suspensions above.

In the examples below, rat CD90⁺ stem cells and/or rat CD90⁺/CD45RA⁺ B cell progenitor cells and/or CD45RA⁺ B cells are employed. Any human bone marrow equivalents of human stem cells and progenitor cells such as, but not limited to, CD34⁺, CD19⁺ cells, or the like, can be employed in the human recipient equivalent of the rat model.

It has unexpectedly been discovered that direct intraosseous implantation of donor derived stem and progenitor bone marrow cells (CD90⁺) into allograft recipients of a CTA from the same donor, in the absence of immunosuppressive therapy and preconditioning, significantly prolonged the survival of the CTA allografts (up to 15 days) compared to recipients receiving the allograft only. This survival extension correlated with a transient chimerism of 3.4% of CD4⁺ T cells of donor origin in the peripheral blood of the recipients. Therefore, implantation of donor stem and progenitor bone marrow cells can produce mixed donor-recipient chimerism, even when the recipient is not given an immunosuppressive regimen.

Enhancement of CTA survival was also evaluated by a comparison of direct intraosseous implantation of donor derived stem and progenitor bone marrow cells and intravenous injection of the same number of cells. Intraosseous delivery of stem and progenitor cells produced a high level of mixed chimerism (1.4% to 5.4%) maintained in the peripheral blood of the recipients for over 35 days post-transplantation. In contrast, intravenous delivery of the same number of stem and progenitor cells produced a transient (up to 5 days) mixed chimerism (2% to 4%). These results confirm the importance of the microanatomic environment and stromal compartment of the bone marrow in the efficacy of donor stem and progenitor cell engraftment. These results further show that direct delivery of the stem and progenitor cells into the bone marrow cavity results in increase efficacy of cell engraftment and augmentation of mixed chimerism, leading to improved graft survival.

A therapeutically effective amount of bone marrow cells is that amount sufficient to induce allograft tolerance and/or the production of mixed donor-recipient chimeric cells in the recipient, and is intended to encompass an amount by weight of crude bone marrow and/or a number of isolated cells or subpopulation of isolated cells in suspension. Preferably, the therapeutically effective amount of the implanted bone marrow cells is an amount sufficient to maintain long-term recipient tolerance of the allograft without the necessity of readministration of the immunosuppressive agent and the anti αβ T cell receptor antibodies. As disclosed in the patent application incorporated by reference above, the level of chimerism present in the peripheral blood of rat hindlimb CTA recipients receiving the combined immunosuppressive treatment and showing indefinite allograft tolerance was about 2% to about 3% of PBMC at about 7 days post transplant. The level of chimerism then rose to about 3% to about 6% at about 21 days post-transplantation, to about 10% at about day 35, and to about 15% to about 20% or more by about day 63 post-transplantation. At this stage, a stable multilineage (CD4, CD8 and CD45RA) chimerism was achieved.

Although in the rat hindlimb model, about 15% to about 20% chimeric cells were sufficient to achieve indefinite allograft tolerance, other protocols in other animals, including human recipients, may achieve a different peripheral blood level of chimeric cells that is sufficient to achieve indefinite allograft tolerance. Thus, an optimum level of chimerism for maintaining long-term allograft tolerance can vary and can be about 5% to about 50% of circulating PBMC, preferably about 10% to about 40%, more preferably about 15% to about 30%, most preferably about 20% to about 30% of circulating PBMC, without limitation, depending on the individual modality employed. The level of chimerism in lymphoid organs can be as high as about 60% or more and as low as about 25% or less.

In another embodiment of the invention, a method is provided for inducing donor-specific tolerance and/or mixed donor-recipient trimerism in an allograft transplant recipient, comprising the steps of: (a) administering to a recipient of an allograft a therapeutically effective amount of an immunosuppressive agent that depletes T cells; (b) administering to the recipient of the allograft a therapeutically effective amount of anti-αβ T cell receptor antibodies; (c) implanting a first allograft from a first allogeneic allograft donor into the recipient; (d) implanting a therapeutically effective amount of bone marrow cells from the first allograft donor into the recipient; (e) implanting a second allograft from a second allogeneic allograft donor into the recipient; and (f) implanting a therapeutically effective amount of bone marrow cells from the second allograft donor into the allograft recipient, wherein a state of trimerism is induced in the recipient. The first and second allogeneic allograft donors are preferably independently selected from semi-allogeneic donors; fully allogeneic donors; and combinations thereof, with respect to the recipient. Preferably, the state of trimerism in the recipient comprises a level of mixed donor and recipient cells of about 0.1% to about 15%, preferably about 1% to about 10%, of circulating peripheral blood mononuclear cells.

This embodiment of the invention is particularly applicable to human transplantation. For example, a patient could receive a solid organ transplant, such as a heart, for example, from one unrelated donor and, later, could receive a kidney from a different unrelated donor. This scenario would produce a state of classical trimerism in the recipient, with co-existing recipient cells, and cells from the donor of the heart and from the donor of the kidney, all co-existing without organ rejection in the recipient. As a further permutation, the same patient could receive another kidney from yet another unrelated donor producing a state in which the cells of the recipient and of three independent donors co-exist in the recipient. This would be a state of “multichimerism.”

In another non-limiting example, a patient could receive a solid organ transplant, such as a kidney, from a related donor that shares one or more MHC loci with the recipient, and a heart transplant from an unrelated donor. This scenario would produce a state of trimerism in the recipient, with cells expressing both donor and recipient antigens co-existing with cells from the related donor. A later transplant from a different related donor that shares a different MHC locus with the recipient, or unrelated donor would produce a state of multichimerism.

In another embodiment of the invention, a method is provided for maintaining a level of mixed donor-recipient chimerism in an allograft transplant recipient, with the goal of prolonging allograft tolerance. By the method, an optimum level of chimerism for maintaining long-term allograft tolerance can be determined by measuring an optimal level of chimeric cells in the recipient not undergoing rejection of the allograft. When an optimal level is achieved, the chimeric cells can be harvested from the recipient and stored or, optionally, propagated in cell culture prior to storage. If the recipient later shows signs of allograft rejection, or if the level of chimeric cells is decreasing, or reaches or falls below a minimum level considered sufficient to maintain allograft tolerance, the harvested stored cells are then available to reconstitute the recipient chimeric cells to maintain the tolerant state.

Optionally, the method can include readministering an effective amount of the immunosuppressive agent and/or the anti-αβ T cell receptor antibodies in addition to the chimeric cells. The harvested stored chimeric cells can be administered to the recipient by any method described above including, but not limited to, direct intraosseous injection into a recipient bone marrow cavity; direct intraosseous injection into a bone marrow cavity of an implanted donor bone allograft; intravenous injection into the recipient; and the like, and combinations of the foregoing.

While individual recipients will vary in the optimal and minimum levels of chimeric cells, the minimum level of mixed donor and recipient cells has been found in the rat model to be about 5% to about 20% of circulating peripheral blood mononuclear cells, and the optimal level has been found to be about 20% to about 50% of circulating peripheral blood mononuclear cells. Individual human recipient equivalents can be established without undue experimentation, and are envisioned to be about the same as in the rat model here described.

The harvested chimeric cells can be obtained from any recipient tissue or lymphatic organ, but are preferably obtained from the peripheral blood. For example, chimeric cells can be obtained by blood donation from allograft recipients on a regular basis such as, but not limited to, monthly, bimonthly, semi-annually, annually, and the like, for any period of time during which optimal levels are maintained.

Peripheral blood mononuclear cells can be separated from the peripheral blood by methods that are well known in the art, and employed for later reconstitution of the recipient. Alternatively, the chimeric cell subpopulations can be separated from the peripheral blood/peripheral blood mononuclear cells by any suitable cell separation method, such as those described above, or the like. Alternatively, the whole peripheral blood can be frozen and stored for later infusion into the recipient.

The harvested PBMC cells and/or chimeric cell subpopulations can be directly stored at −196° C. in liquid nitrogen, or by similar known means. Preferably, the cells are stored in a medium suitable for cryogenic preservation. Alternatively, the harvested cells can undergo expansion in culture prior to storage, using appropriate culture media, feeder cell layers, and the like, that will allow proliferation of the cells. For example, a suitable method for culturing harvested rat cells employs a MyeloCult medium (StemCell Technologies, Vancouver, BC) and rat cell feeder layers in tissue culture dishes. The harvested chimeric cells can be divided and a portion of the harvested cells used to reconstitute the recipient, with the remaining portion stored for later use.

### EXAMPLES

To illustrate embodiments of the method of the invention, the following examples employ several allograft transplantation models, immunosuppressive protocols, and delivery systems for donor bone marrow and/or bone marrow cells, including isolated donor stem and progenitor cell populations The examples described herein are not intended to be limiting, as one skilled in the art would recognize from the teachings hereinabove and the following examples that, for example, other immunosuppressive agents, other anti-αβ T cell receptor antibodies, other dosage and treatment schedules, other methods of bone marrow delivery, other isolation methods for donor stem and progenitor cell recovery, other sources of donor stem and progenitor cells, other animal and/or humans, and the like, all without limitation, can be employed, without departing from the scope of the invention as claimed.

### Example 1

Extended Survival of Allogeneic Skin Transplants in Recipients Under a Combined CsA/αβTCR mAb Immunosuppressive Treatment Protocol and Direct Transplantation of Crude Donor Bone Marrow Into Recipient Bone Marrow Cavity

In this example, 43 skin graft transplantations were performed in 9 animal groupings, described below, between isogeneic [Lewis to Lewis (LEW, RT-1¹)] and semi-allogeneic [Lewis x Brown Norway (LBN→F1, RT-1¹⁺ⁿ) to Lewis] rat strains under anti-αβ-TCR mAb and CsA treatment. The allogeneic skin graft recipient also received a crude bone marrow transplantation from the same donor into a recipient bone marrow cavity. The use of combined protocol of CsA/αβ-TCR mAb and the crude bone marrow transplantation resulted in the extension of skin allograft survival up to 65 days after cessation of the immunosuppressive treatment (p<0.05).

The following animals, reagents, assays and techniques were employed in Example 1.

I. Animals

Inbred male rats weighting 150-175 grams were purchased from Harlan Sprague-Dawley, Indianapolis, Ind.). Lewis rats (LEW) served as the recipients of skin and crude bone marrow allografts from Lewis-Brown Norway donors (LBN). The animals were caged at room temperature on a 12-hour light/dark cycle with free access to food and water. All animals received humane care in compliance with the Guide for the Care and Use of Laboratory Animals published by the National Institute of Health in the facility accredited by the American Association for the Accreditation of Laboratory Animal Care.

II. Transplantation Techniques

Skin grafting and crude bone marrow transplantation were performed at the same session from the same donor. Intraperitoneal pentobarbital (50 mg/kg) was used as an analgesic during the transplantation procedure.

A. Skin Grafting Transplantation Technique

Skin grafting was performed according to the technique described by Billingham (Billingham R E. and P. B. Medawar. J. Ex. Biol. 1951; 28: 385-402). Briefly, full thickness skin grafts 16 mm in diameter were taken from the donors. Graft beds were prepared by excising 18 mm circles on the lateral dorsal thoracic walls of the recipients. Care was taken to remove perniculous carnosum from the grafted skin. Both sides of the thoracic wall were used for allogeneic grafts and the mid-sternum was used for syngeneic grafts. All grafts were separated by a 10 mm skin bridge. A standard compressive dressing and adhesive bandage was used for 7 days.

B. Crude Bone Marrow Transplantation Technique

Donor-origin bone marrow was transplanted in its crude form containing all natural microanatomical components of the bone marrow including pluripotent stem cells and progenitor cells and extracellular matrix.

(a). Harvesting of the crude bone marrow from the donor: The metaphyseal region of the right tibia was approached from an anterior incision. Next, on the anteromedial cortex of the tibia, a 3 mm window was created using a 1/32-inch drill. Following decortication, the contents of the metaphyseal part of the bone were removed with a bone curette and placed in a container cooled in ice. Next, the bone rongeur was used to harvest intramedullary content along the diaphysis. The weight of the harvested BM was measured before transplantation.

(b). Transplantation of the crude BM to the recipient: The recipient's tibia was opened in a similar fashion as the donor's bone, exposing the anteromedial surface of the right tibia, followed by creation of the cortical window. The recipient's cancellous bone and content of the BM cavity were totally removed to create an appropriate “space” for the BM engraftment. The harvested bone marrow of donor origin was next packed into the “empty” medullary cavity of the recipient's tibia and sealed with bone wax.

III. Immunosuppressive Treatment Protocols

Treatment protocols included monotherapy with CsA alone or anti-αβ TCR monoclonal antibody (anti-αβ-TCR mAb) alone, or a combination of CsA and anti-αβ TCR monoclonal antibody (CsA/αβ-TCR mAb). Both CsA and anti-αβ-TCR mAb were administered 12 hours before transplantation and continued up to 7 days or 35 days in the combined therapy group.

Cyclosporine A (Sandoz Pharmaceutics Inc., East Hanover, N.J.) was dissolved daily in PBS (Fisher Scientific, Pittsburgh, Pa.) to a concentration of 5 mg/ml and administered subcutaneously (s.c.) to recipient animals. Animals under CsA treatment received a dose of 16 mg/kg/day (s.c.) administered 12 hours before transplantation and daily thereafter for the first week, 8 mg/kg/day during the second week, 4 mg/kd/day for the third and fourth week, and 2 mg/kg/day for the fifth week. Intraperitoneal (i.p.) injection of anti-αβ TCR mAb (clone R73, Pharmingen, San Diego, Calif.) (250 μg) was administered 12 hours before transplantation, and daily thereafter for the first week. The dosage of anti-αβ TCR mAb was then tapered to 50 μg at the end of the first week and was given every 2 days during the second week and every 3 days during the last 3 weeks.

The 7-day protocol was similar. Animals under CsA treatment received a dose of 16 mg/kg/day (s.c.) administered one hour or 12 hours before transplantation (when semi-allogeneic transplants were performed) and daily thereafter for 7 days. Intraperitoneal injection of anti-αβ TCR mAb (250 μg) was administered one hour or 12 hours before transplantation (when semi-allogeneic transplants were performed), and daily thereafter for 7 days.

In each of the foregoing protocols, when fully allogeneic transplants were performed, both the CsA and anti-αβ TCR mAb treatments were initially administered at the time of transplantation, at the time of clamp release.

IV. Treatment Groups

The treatment groups, treatment protocols, and graft survival are illustrated in Table 1. Multiple trials were performed according to the protocols. The immunosuppressive treatment was administered using the 35 protocol or the 7 day protocol. The amount of crude donor bone marrow transplanted ranged from 20 mg to 100 mg.

The following treatment groups were employed in one series of transplants between semi-allogeneic donors/recipients:

Group 1: Skin Isograft Control (n=6): Skin grafts were transplanted between LEW rats without immunosuppressive treatment before or after transplantation.

Group 2: Skin Allograft Control (n=6): Skin allografts were transplanted from LBN donors to LEW recipients without immunosuppressive treatment before or after transplantation.

Group 3. Skin and Bone Marrow Allograft Control (n=6): Both the skin and the crude bone marrow were transplanted from donor LBN to recipient LEW rats without immunosuppressive treatment before or after transplantation.

Group 4. Skin Allografts+CsA (n=3): Skin allografts were transplanted from LBN donor to LEW recipients. Animals in this group received 5 weeks of CsA treatment following skin transplantation.

Group 5. Skin Allografts+anti-αβ TCR mAb (n=3): Skin allografts from LBN donors were transplanted to LEW recipients. Animals received 5 weeks of anti-αβ TCR mAb treatment following skin transplantation.

Group 6. Skin Allograft+CsA+anti-αβ TCR mAb (n=5): Skin allografts from LBN donors were transplanted to LEW recipients. Animals received 5 weeks of combined CsA and anti-αβ TCR mAb treatment following skin transplantation.

Group 7. Skin Allograft+BM+CsA (n=5): Skin and the crude bone marrow from the same LBN donors were transplanted to LEW recipients. The recipient received 5 weeks of CsA treatment following transplantation.

Group 8. Skin Allograft+BM+anti-αβ TCR mAb (n=4): Skin and the crude bone marrow from the same LBN donors were transplanted to LEW recipients. The recipient received 5 weeks of anti-αβ TCR mAb treatment following transplantation.

Group 9. Skin Allograft+BM+CsA+anti-αβ TCR mAb (n=5). Combined CsA and anti-αβ TCR mAb protocol was applied for 5 weeks following the skin and the crude bone marrow allograft transplantation from the same LBN donors to LEW recipients.

V. Clinical Assessment of Allograft Rejection

The physical signs of skin allograft rejection, such as erythema, edema, loss of hair, scaling of the skin, and desquamation were evaluated on a daily basis. Rejection was defined as the destruction of over 80 percent of the graft.

VI. Assessment of Graft Versus Host Disease (GVHD)

All animals were evaluated for the appearance of GVHD. The clinical criteria, such as diffuse erythema (particularly of the ear), hyperkeratosis of the foot pads, dermatitis, weight loss, generalized unkempt appearance, or diarrhea were monitored daily. An animal was considered to exhibit acute GVHD if at least four of the above signs were observed. The diagnosis of GVHD was confirmed by routine hematoxylin and eosin histologic staining performed on formalin-fixed skin, tongue, liver and small intestine samples collected at the time of occurrence of the first signs of GVHD. Grading of GVHD was performed in blinded fashion according to previously described histologic criteria (Sale, G. E. et al Am. J. Surg. Pathol. 1979; 3: 291; Saurat, J. H. et al. Br. J. Dermatol. 1975; 93: 675) and were assessed by a histopathologist.

VII. Flow Cytometry Analysis

Flow cytometry (FC) analysis was performed according to the manufacturer's protocol (Becton Dickinson, San Diego, Calif.) with minor modifications. The blood samples of transplant recipients were collected into heparinized tubes on the following days post-transplantation: 0, 7, 21, 35, 63 and at the time of initial signs of clinical rejection. The peripheral blood mononuclear cells (PMBC) were incubated for 20-30 minutes in the dark at room temperature with 5 μL of a mixture of mouse anti-rat monoclonal antibodies conjugated with fluorescein isothiocyanate (FITC) or phycoerythrin (PE) against CD4^{-FITC} (Clone OX35), CD8a^{-PE} (Clone OX8), αβ TCR^{-FITC} (Clone R73), CD45RA^{-PE} (Clone OX-33). After incubation, samples were resuspended in FACS Lysing solution (Becton Dickinson), incubated for 20 minutes in the dark at room temperature, centrifuged at 1500 rpm for 5 minutes, washed twice with washing buffer (phosphate-buffered saline, PBS, without Mg⁺⁺, Ca⁺⁺, 0.1% bovine serum albumin, 0.05% NaN₃), fixed with 2% paraformaldehyde solution, covered with aluminum foil and stored at 4° C. until flow cytometry assessment.

VIII. Donor-Specific Chimerism Evaluation by FC

For the determination of donor-specific lymphoid chimerism in the peripheral blood of recipients, combinations of mouse anti-rat CD4^{-PE} or CD8a^{-PE} with RT-1ⁿ (Brown Norway MHC class I, Clone MCA-156, Serotec, Kidlington, UK) were applied. For the donor-derived, RT-1ⁿ-positive staining cells, purified anti-rat CD-32 (FcyII Block Receptor) antibody (1:20) was added first to block the Fc-mediated adherence of antibodies. After 3-4 minutes pre-incubation, samples were further incubated with 5 μL of mouse anti-rat RT-1ⁿ for 30 minutes at 4° C. Then samples were washed twice in washing buffer and stained with goat anti-mouse FITC conjugated IgG (rat adsorbed; Serotec). This was followed by the incubation with CD4^{-PE} or CD8^{-PE} conjugated mouse anti-rat monoclonal antibody. After incubation, samples were processed as described above. The negative control included isotype-matched antibodies and/or PBS-incubated samples. FC analyses were performed on 1×10⁴ mononuclear cells by using FACS Scan (Becton Dickinson) and CellQuest software.

IX. Statistical Evaluation

Treatment groups were compared on survival using the log-rank test, and survival times were estimated using the Kaplan-Meier method. The level of chimerism, and efficacy of the immunosuppressive treatment were compared by the independent samples t test. Differences were considered statistically significant at p<0.05.

The Survival of the Skin Allografts. The survival of skin allografts in each transplant group is presented in Table 1. The combined protocol of CsA and anti-αβ TCR mAb applied to the recipients of skin allografts without bone marrow significantly extended allograft survival when compared to the allograft controls without treatment (p<0.05). However, following cessation of Cs/αβ TCR mAb therapy, all allografts were rejected within 20 days (p<0.05). It was demonstrated that monotherapies, combined with crude donor bone marrow transplantation, resulted in extended survival up to 21 days (under CsA) and up to 10 days (under anti-αβ-TCR mAb). Monotherapy with CsA alone extended skin transplant survival over 7 days, whereas monotherapy with anti-αβ TCR mAb alone resulted in rejection at the same time as the rejection controls without treatment. When skin allograft transplantation and the crude bone marrow transplantations were performed in one procedure, the skin allograft transplant survival was extended by over 60 days (p<0.05) after cessation of the CsA/αβ TCR mAb protocol.

Extended skin allograft survival was achieved in animals receiving crude bone marrow under short term (7 days only) and longer term (5 weeks) CsA/αβ-TCR mAb treatment protocol.

Determination of GVHD. None of the allogeneic transplant recipients showed clinical signs of GVHD.

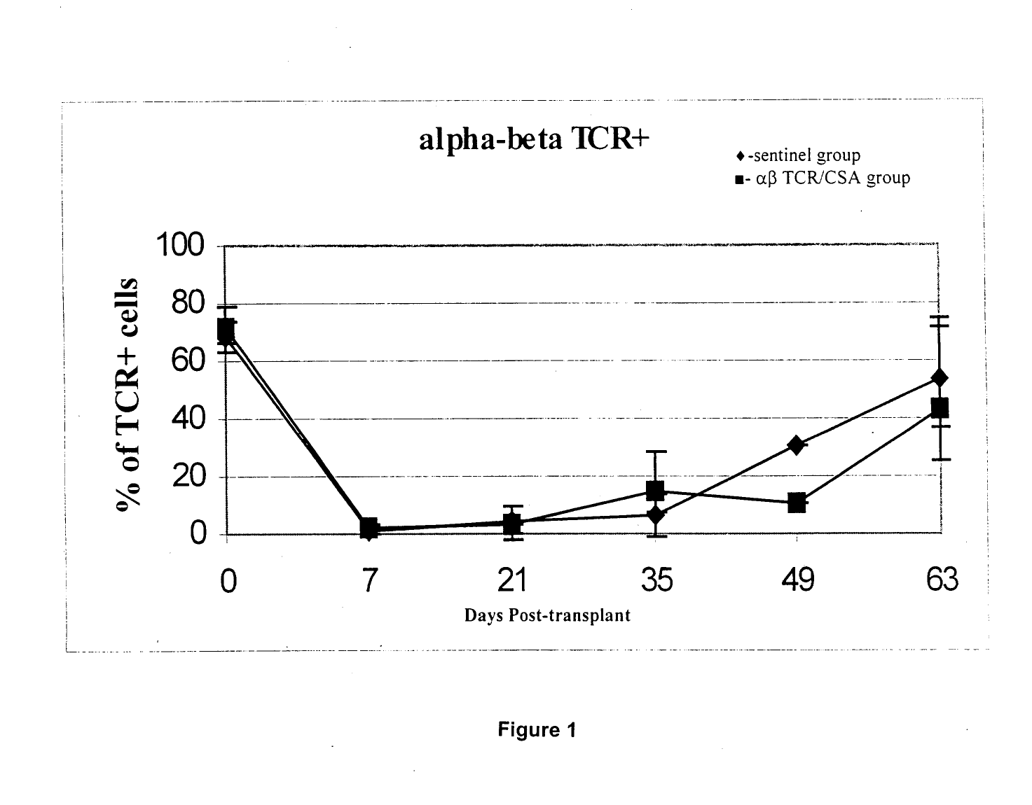

Flow Cytometry Analysis of In Vivo Depletion of αβ TCR⁺ Cells. As illustrated in FIG. 1, FC determination of αβ TCR expression on lymphocytes harvested from sentinel (untransplanted animals treated with CsA/αβ TCR mAb) and transplanted animals treated with CsA/αβ TCR mAb showed >90% depletion of the αβ TCR⁺ cell population 7 days after immunosuppressive treatment cessation (day 42 post-transplantation). Repopulation of αβ TCR⁺ cell populations to the pre-transplantation level was observed 28 days after cessation of the immunosuppressive treatment (day 63 post-transplantation). The population of CD4+ and CD8⁺ cells in the peripheral blood of the sentinel and transplanted recipients was significantly reduced at day 7 (75% of CD4⁺ and 55% of CD8⁺) and a gradual repopulation of both cell subpopulations was seen at day 63 (data not shown).

TABLE 1

| | Transplantation | Immunosuppressive | Days of Survival After | |
|---|---|---|---|---|
| Transplant Groups | Procedure¹ | Treatment Protocol | Cessation of Treatment | Mean ± SD |
| Group 1 (n = 6) Isograft | Skin | None | Indefinite | Indefinite |
| Group 2 (n = 6) Allograft | Skin | None | 6, 8, 7, 6, 7, 8 | 7 ± 0.9 |
| Group 3 (n = 6) Allograft | Skin | None | 7, 8, 9, 6, 7, 8 | 7.5 ± 1 |
| Group 4 (n = 3) Allograft | Skin | CsA | 15, 16, 15 | 15 ± 0.5 |
| Group 5 (n = 3) Allograft | Skin | aβTCR mAb | 7, 9, 10 | 8.6 ± 1.5 |
| Group 6 (n = 5) Allograft | Skin | CsA + aβTCR mAb | 20, 21, 20, 19, 22 | 20.4 ± 1.1 |
| Group 7 (n = 5) Allograft | Skin + Crude BM | CsA | 20, 22, 24, 21, 20 | 21.4 ± 1.7 |
| Group 8 (n = 4) Allograft | Skin + Crude BM | aβTCR mAb | 9, 12, 10, 11 | 10.5 ± 1.3 |
| Group 9 (n = 5) Allograft | Skin + Crude BM | CsA + aβTCR mAb | 60, 65, 60, 67, 71 | ^{65 ± 4.7}² |

¹All tranplantations except in group 1 were performed with LBN donors and LEW recipients.
²p <0.05

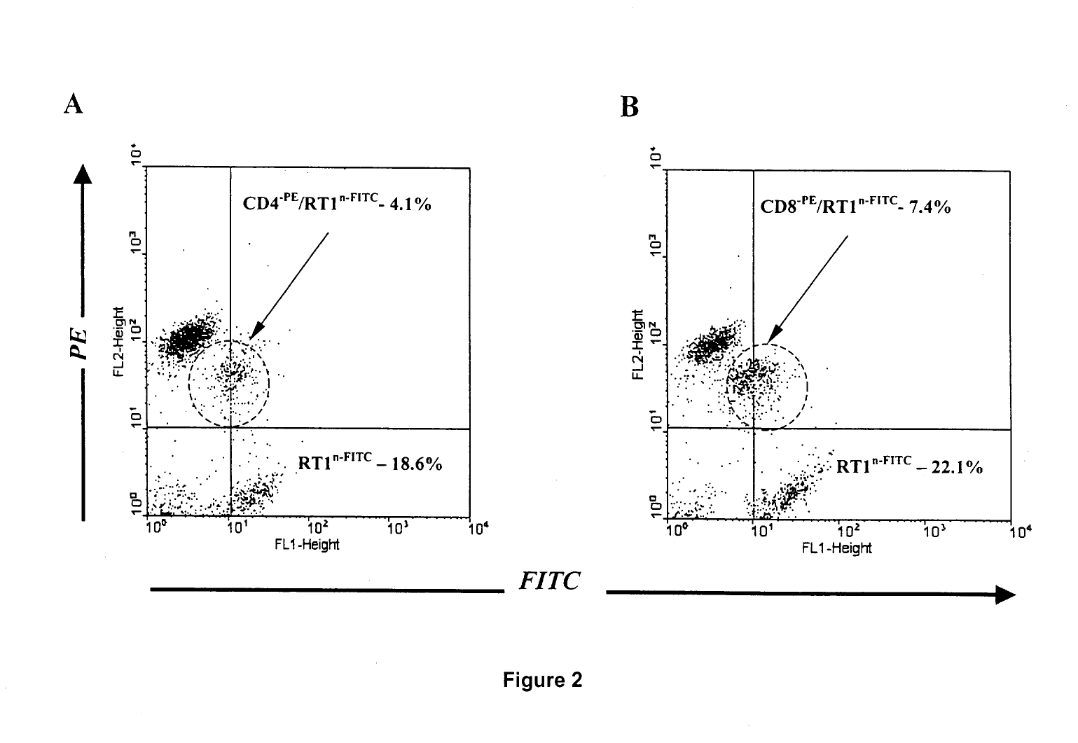

Mixed Donor-Recipient Chimerism in Recipients of Crude Donor Bone Marrow. Two-color flow cytometry analysis of the donor-specific macro-chimerism was performed on PBMC of recipients of crude donor bone marrow and skin allografts. The dot-plot results of the lymphoid cell subpopulations obtained from quadrangle analytical gates at day 65 after allotransplantation demonstrated the presence of the multilineage donor-specific chimerism ranging from 18-29% (RT-1ⁿ positive cells). Examination of two-color stained RT-1^{n-FITC}/CD4^{-PE} and RT-1^{n-FITC}/CD8^{-PE} peripheral lymphocytes revealed 4.1% and 7.4%, respectively, of double positive CD4 and CD98 T cell subpopulations, illustrated in FIG. 2, A and B, respectively. The expression of RT-1ⁿ antigen on non-CD4 and non-CD8 positive T cell populations suggests the existence of donor/recipient chimeric residents not evaluated with the PE-conjugated mAb against surface specific antigen (B-lymphocytes and monocytes). Therefore, the donor crude bone marrow transplant inoculated directly into the recipient's bone marrow cavity allowed for optimal engraftment, repopulation and subsequent trafficking outside the bone marrow cavity into the periphery, resulting in donor specific chimerism.

In trials employing different amounts of crude donor bone marrow ranging from 20 mg to 100 mg, it was found that chimerism and extended allograft survival was achieved when the weight of transplanted crude bone marrow was greater than about 50 mg.

### Example 2

Enhancement of CTA Survival by Intraosseous Delivery of Donor-Derived Stem Cells and Progenitor Cells

This example illustrates that increases in the level of hematopoietic chimerism can improve the survival and maintenance of CTA transplants without immunosuppressive therapy. The exemplary design included transplantation of the rat hindlimb allograft (LBN to LEW) concomitant with the direct intraosseous transplantation of bone marrow stem and progenitor cells isolated from the same donor. The following techniques and treatment regimens were employed.

I. Hindlimb Transplantation Technique

Transplantations of hindlimbs between donor and recipient were performed under pentobarbital (50 mg/kg intraperitoneal) anesthesia using a standard microsurgery procedure (Press, B. H. J. et al. 1986. Ann. Plast. Surg. 16: 313-321). Briefly, a circumferential skin incision was made in the proximal one third of the right hindlimb. The femoral artery and vein were dissected, clamped, and cut proximal to the superficial epigastric artery. The femoral nerve was dissected and cut 1 cm distal to the inguinal ligament. The biceps femoris muscle was transected to expose the sciatic nerve. The nerve was then cut proximal to its bifurcation.

The donor was prepared in a similar way. The right hindlimb was amputated at the midfemoral level. The donor limb was attached to the recipient limb by a 20-gauge intramedullary pin and a simple cerclage wire. All large muscle groups were sutured in juxtaposition. The iliac vessels of the donor and femoral vessels of the recipient were anastomosed under an operating microscope with 10-0 sutures by using a standard end-to-end microsurgical anastomosis technique. The femoral and sciatic nerves were repaired by using a conventional epineural technique with four 10-0 sutures.

II. Purification of CD90⁺ Stem and Progenitor Bone Marrow Cells

Bone marrow cells were isolated from donors using flushing methods. Briefly, freshly isolated femur and tibia were washed with sterile, cold PBS (without Mg⁺⁺ and Ca⁺⁺) supplemented with 1.0% bovine serum albumin (BSA). Two contralateral ends of the bones were cut and residual bone marrow cells were flushed out from the bone marrow cavity using a syringe-based pump system. After lysis with NH₄Cl/TRIS sterile hemolytic buffer for 5 minutes, nucleated marrow cells were then washed (PBS, 1.0 BSA) twice and counted to obtain a final concentration of 1×10⁶ nucleated cells/ml.

For isolation of the CD90⁺ cells, isolated nucleated bone marrow cells were incubated with FITC conjugated mouse anti-rat CD90 mAb (OX-7, Pharmingen) for 30 minutes in the dark at 4° C. After incubation, samples were washed twice with washing buffer, incubated for 30 minutes in the dark at 4° C. with magnetic beads-conjugated mouse anti-FITC mAb, washed, and placed into MiniMACS separation columns (Miltenyi Biotec, Auburn, Calif.). The CD90⁺ cells were collected and their viability and number assessed by counting of the cells incubated with trypan blue. The assessment of the efficacy of purification of the MACS positively selected cells was accomplished by FC evaluation of the level of double positive CD90^{FITC} (Clone OX7)/RT-1ⁿ cells.

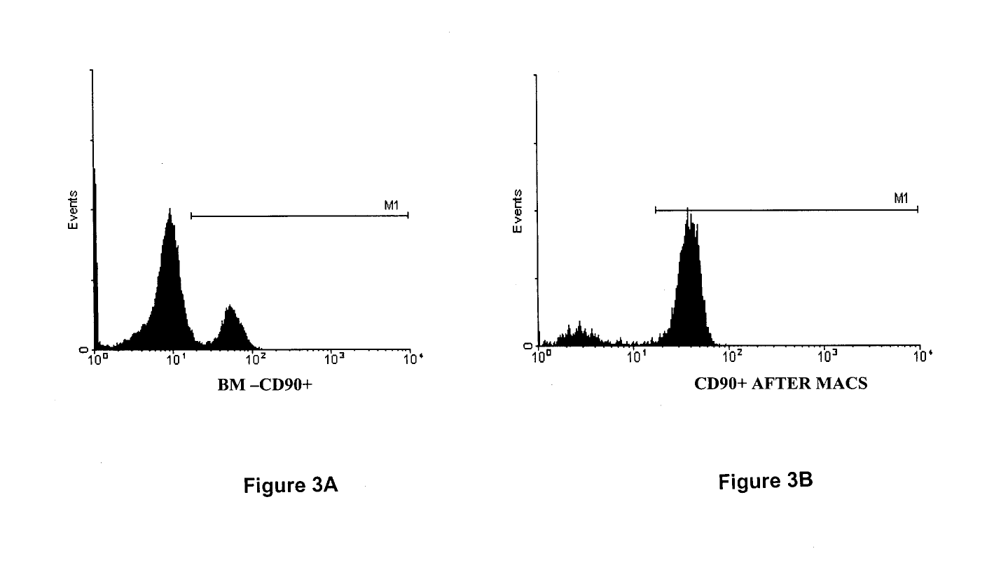

FIG. 3 illustrates FC analysis of the CD90⁺ antigen expression on the surface of stem and progenitor cells from donor bone marrow before and after selection using a magnetic MiniMACS separating system. FIG. 3A shows CD90⁺ cells as the small peak, and the remainder of the bone marrow nucleated cells as the larger peak, prior to selective separation of the CD90⁺ cells from a suspension of bone marrow cells. The CD90⁺ cells comprised about 22% of the bone marrow nucleated cells. FIG. 3B shows the purity of separation of the CD90⁺ cells (large peak). Less than 5% were CD90⁻. Over 95% of analyzed cells expressed the CD90 antigen, indicating high efficacy of the selection.

III. Intraosseous Injection of Donor Derived Stem and Progenitor Cells

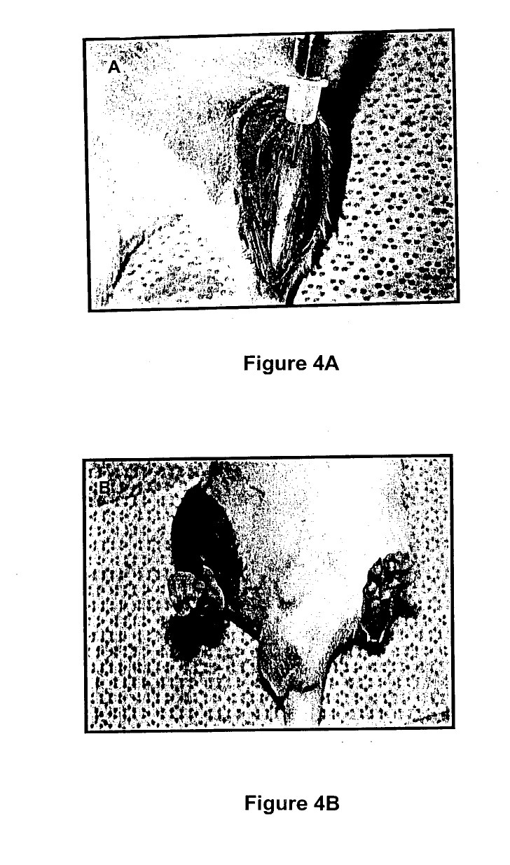

A 50 μL high-purity suspension containing 8-12×10⁵ CD90⁺ stem and progenitor cells was injected directly into the bone marrow cavity of the recipient's contralateral tibia just before transplantation of the opposite hindlimb. No immunosuppressive protocol was given. FIG. 4A illustrates injection of the stem and progenitor cells into the bone marrow cavity of the recipient's left tibia. Following injection, the right hindlimb from the same donor was transplanted to the recipient. (FIG. 4B)

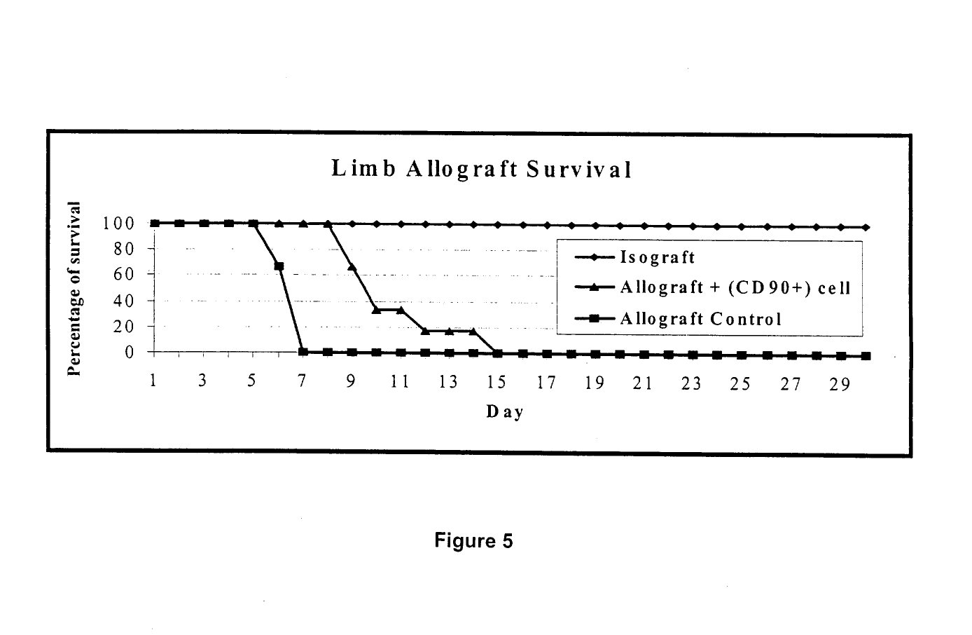

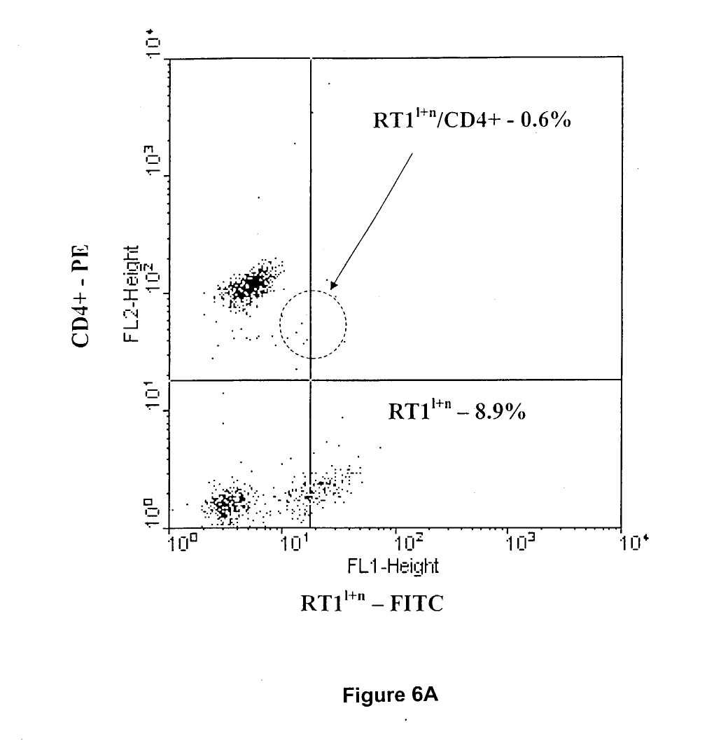

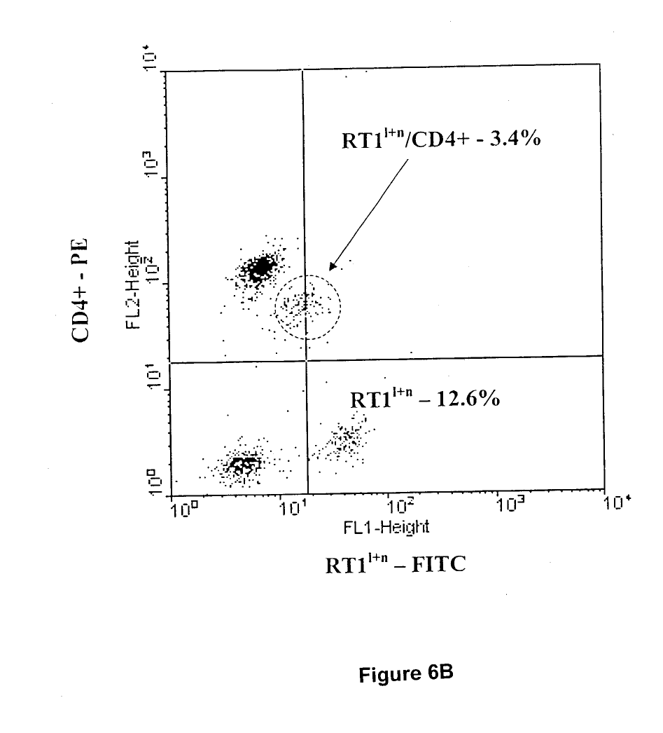

Survival of Limb Allografts Following Preooperative Intraosseous Injection of Donor Stem and Progenitor Cells. As shown in the limb allograft survival chart illustrated in FIG. 5, recipients receiving the allograft and also receiving intraosseous injection of donor CD90⁺ stem and progenitor cells, had a significant biological extension (up to 15 days) of limb allograft survival without immunosuppressive therapy (p<0.05), compared to recipients receiving the allograft only. This survival extension correlates with a transient chimerism level of 3.4% of CD4⁺ T cells of donor origin in the peripheral blood of the recipients, illustrated by the FC analysis of FIG. 6. A two-color FC analysis at the 14^{th} day after limb allograft transplantation revealed transient chimerism in the allograft rejection control group without treatment (0.6%, FIG. 6A) and a higher level (3.4%, FIG. 6B) of double positive RT-1¹⁺ⁿ/CD4⁺ chimeric cells in the peripheral blood of the limb recipients treated with the intraosseous injection of the CD90⁺ stem and progenitor cells at the time of transplantation.

### Example 3

This example illustrates a comparison of the level of donor/recipient chimerism in the hindlimb transplantation model, after intraosseus transplantation of donor stem and progenitor cells or intravenous injection of the same number of donor stem and progenitor cells.

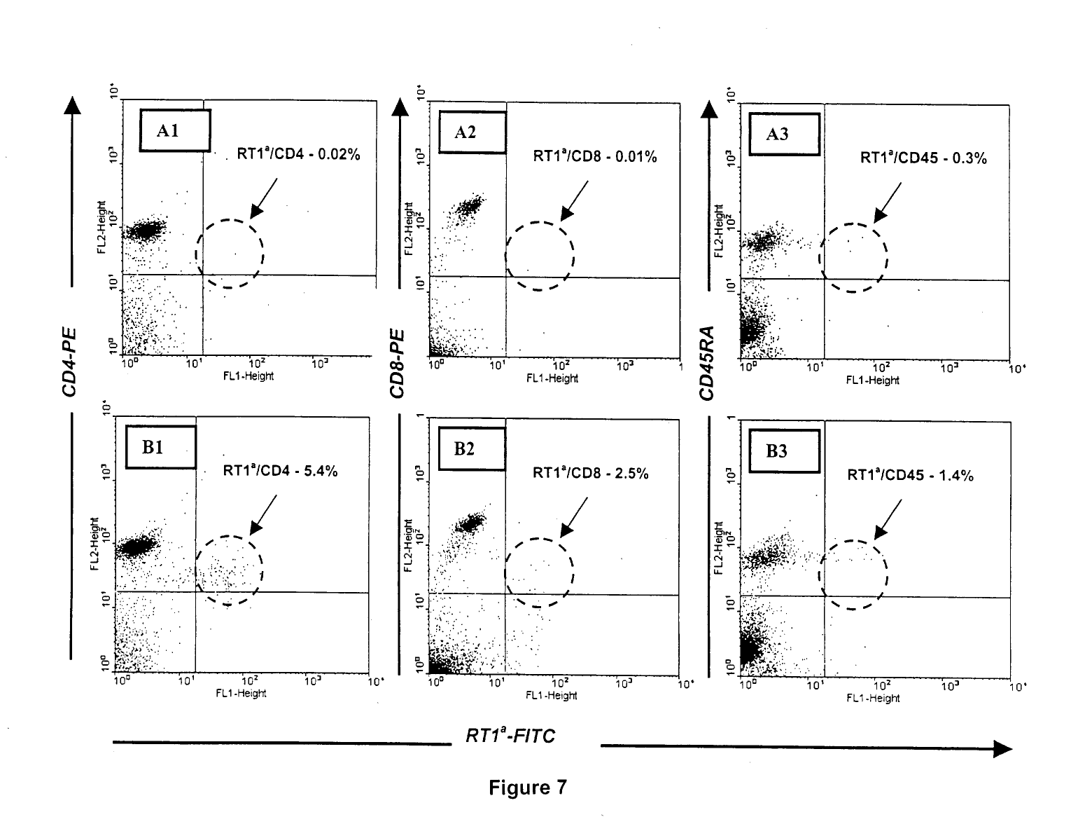

Intraosseous Transplantation of Donor Stem and Progenitor Cells Produces Long Term Donor-Specific Chimerism. In a further example, 50 μL of a high purity (>95%) suspension containing 35-40×10⁶ donor stem and progenitor cells were obtained as described above. The same number (35-40×10⁶) of cells was injected intravenously into the epigastric vein in one group of recipient rats and directly into the tibial bone marrow cavity in the another group of recipient rats. Hindlimb transplants were then performed as described above. At day 35 post-transplant, flow cytometry revealed high levels of multilineage donor-specific lymphoid chimerism (FIG. 7, B1-B3) in the peripheral blood of the recipients receiving direct intraosseous donor stem and progenitor cells at the time of transplantation, which was maintained over 35 days (still under evaluation). In contrast, intravenous injection of the donor stem and progenitor cells at the time of transplantation resulted in low-level, transient (up to 5 days) chimerism (FIG. 7, A1-A3). These results confirm the importance of the microanatomic environment and stromal compartment in the efficacy of stem and progenitor cell engraftment. The results show that direct delivery of the stem and progenitor cells into the bone marrow cavity results in increased efficacy of donor cell engraftment and augmentation of mixed chimerism.

### Example 4

Chimerism in Vascularized Skin/Vascularized Bone Transplantation

A vascularized embodiment of CTA comprising vascularized skin with subcutaneous fat (VS), and vascularized bone (VB) with cartilage and bone marrow was employed. The allograft transplantations were carried out across MHC semi-mismatched donors and recipients (LBN; RT-1¹⁺ⁿ→LEW; RT-1¹) and MHC fully mismatched donors and recipients (BN; RT-1ⁿ→LEW; RT-1¹), as illustrated below. Ten animals were employed in each group. The treatment protocols and surgical procedures are also described below.

| Type of Graft | Donor | Recipient |
|---|---|---|
| Isogeneic graft | Lewis (LEW; RT-1¹) | Lewis (LEW; RT-1¹ |
| Semi-allogeneic graft | Lewis Brown Norway | Lewis (LEW; RT-1¹) |
| | (LBN; RT-1¹⁺ⁿ) | |
| Fully allogeneic graft | Brown Norway | Lewis (LEW; RT-1¹) |
| | (BN; RT-1ⁿ) | |

I. Immunosuppressive Treatment Protocol

All recipients of different combinations of the vascularized skin and (vascularized) bone allograft (VSBA) transplants were given the immunosuppressive therapy, whereas isograft controls received no treatment. A 7-day immunosuppressive treatment protocol, similar to that described in Example 1, was employed. Briefly, the treated animal groups received 16 mg/kd/day s.c. of CsA and 250 μg/day i.p. of anti-αβ TCR mAb daily for 7 days. The first treatments were given one hour prior to transplantation. Isogeneic transplant recipients received no immunosuppressive therapy.

II. Surgical Procedures

A. Vascularized Skin Grafting Procedure

Vascularized skin allograft transplantation was performed according to the technique described by Strauch et al. (Strauch, B. and D. E. Murray. Plast. Recons. Surg. 1967; 40: 325-329). Briefly, a standard 4×6 cm template was used to mark the flap borders both in the donor and the recipient. The donor skin flap was elevated on the superficial epigastric branch of the femoral artery and vein of the donor, and end-to-end anastomoses were performed between the donor's and recipient's femoral arteries and veins using standard microsurgical techniques.

B. Vascularized Bone Transplantation

A vascularized femoral bone allograft was harvested on the femoral artery and vein of the donor, preserving supplying collateral vessels. The bone allograft was transferred to the recipient's groin region and end-to-end anastomoses between donor's and recipients femoral arteries and veins were performed using standard microsurgical techniques.

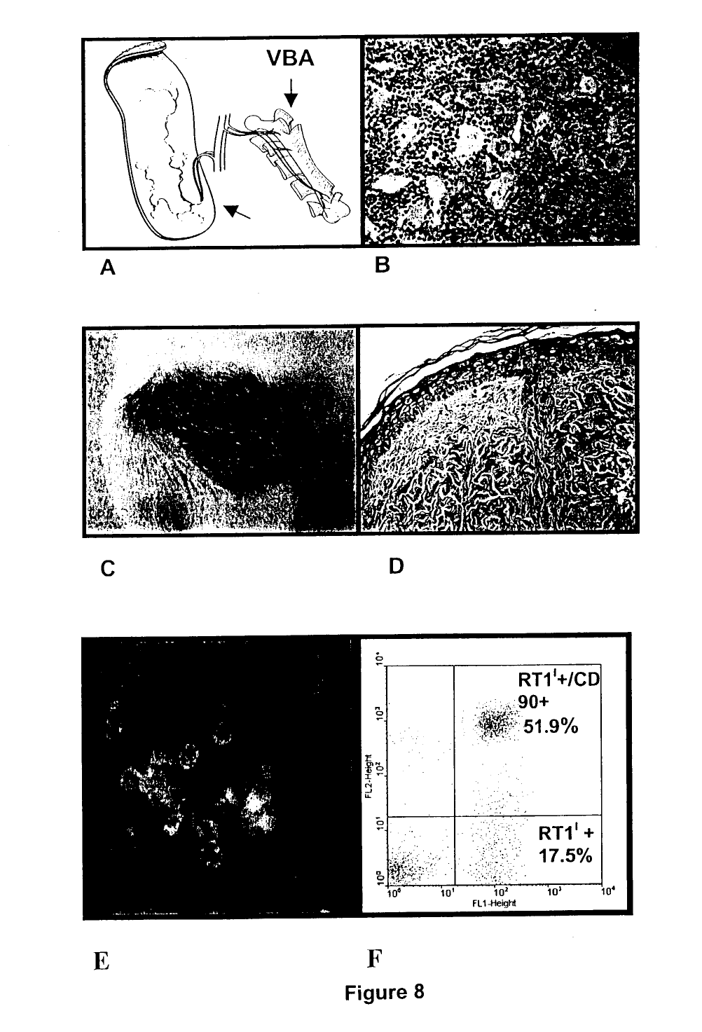

FIG. 8A is a schematic representation of the vascularized skin and bone allograft (VSBA).combining superficial epigastric skin flap and vascularized femoral bone allograft.

VSBA Transplants are Accepted Across Semi-Allogeneic and Fully-Allogeneic MHC Barriers. The VSBA isografts, and semi-allogeneic and fully allogeneic grafts in recipients receiving the combined immunosuppressive therapy, showed indefinite (over 200 days) survival of both the skin and bone components of this tissue assembly, whereas VSBA allograft controls without immunosuppressive therapy rejected uniformly within 7 days (not shown). For example, FIG. 8B illustrates a Giemsa stained (donor) vascularized bone marrow isograft 7 days after transplantation into the recipient, showing over 99% viability of the bone marrow cells in the bone transplants. FIG. 8C shows complete acceptance of a vascularized skin allograft in a representative fully allogeneic VSBA recipient transplanted across a major MHC barrier (BN→LEW) at day 63 after cessation of immunosuppression. Biopsy of the skin (hematoxylin and eosin stained) shows preserved dermis and epidermis and no histological signs of rejection (FIG. 8D).

Trafficking of Bone Marrow-Derived Cells From VSBA Transplant Recipients to Donor Bone Marrow. FIGS. 8E and 8F show immunohistostaining of frozen sections of the bone marrow tissue, and FC analysis of the bone marrow cells, respectively, taken from the donor vascularized bone transplant in the VSBA of a representative fully allogeneic recipient at day 63 after cessation of immunosuppression. The analysis showed replacement of the CD90⁺ stem cells of the donor by the recipient's CD90⁺ cells. More than 50% of the cells were CD90⁺ and RT-1^{L} (related to LEW MHC class I) positive cells of the recipient origin. These results proved the trafficking of the donor bone marrow derived stem cells between the recipient and donor bone and confirmed the viability of the transplanted vascularized bone marrow. Similar results were obtained in semi-allogeneic transplants (data not shown).

### Example 5

Development of a Trimeric Recipient from Fully Allogeneic Transplants from Genetically Unrelated Donors

The VSBA transplantation surgical procedure was performed as described in Example 4. Each Lewis (LEW, RT-1¹) rat received a vascularized bone allograft, including a vascularized skin flap, from two genetically unrelated allograft donors, i.e., Brown Norway (BN, RT-1ⁿ) and ACI (A x C Irish, RT-1^{a}) rats. Both VSBA transplantations were performed during the same operative procedure.

Control LEW recipients received a vascularized skin allograft alone, without the vascularized bone component, from both fully allogeneic donors.

All LEW recipient received a 7-day immunosuppressive treatment protocol, described in Example 1. Briefly, the treated animal groups received 16 mg/kd/day s.c. of CsA and 250 μg/day i.p. of anti-αβ TCR mAb daily for 7 days. The initial treatments were given at the time of transplantation at the time of clamp release

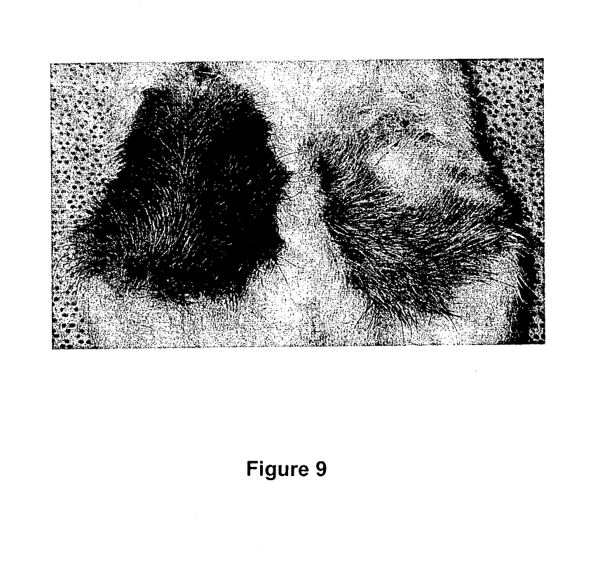

Two Genetically Unrelated VSBA Transplants are Accepted Across Fully-Allogeneic MHC Barriers, Producing a Fully Tolerant Trimeric Recipient. FIG. 9 illustrates a LEW recipient of two genetically unrelated VSBA transplants at day 35 after transplantation. The vascularized bone transplants are not visible, as they are beneath the transplanted skin flaps shown. On the left is a VSBA transplant from a BN donor, showing full skin acceptance by the LEW recipient of this fully allogeneic transplant. On the right is a VSBA transplant from an ACI donor, showing full skin acceptance by the LEW recipient of this fully allogeneic transplant.

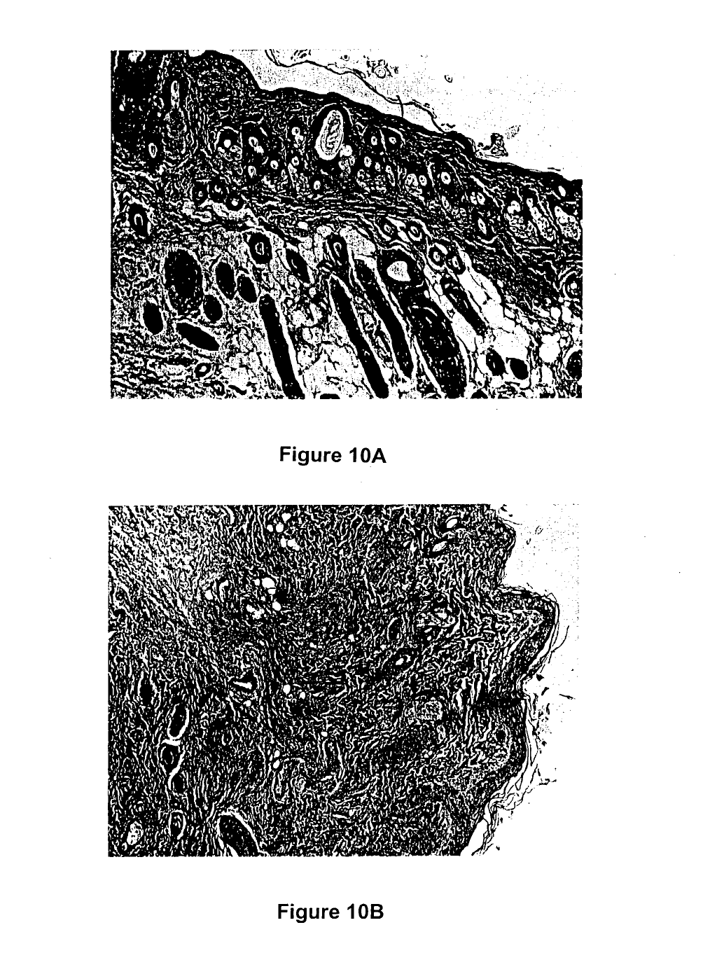

FIGS. 10A and 10B illustrate H&E stained formalin-fixed skin tissues taken from the BN allograft and the ACI allograft, respectively, at day 21 after transplantation, showing preserved dermis and epidermis and no histological signs of rejection. At over 100 days after transplantation (to the present), neither of the skin grafts show any signs of rejection.

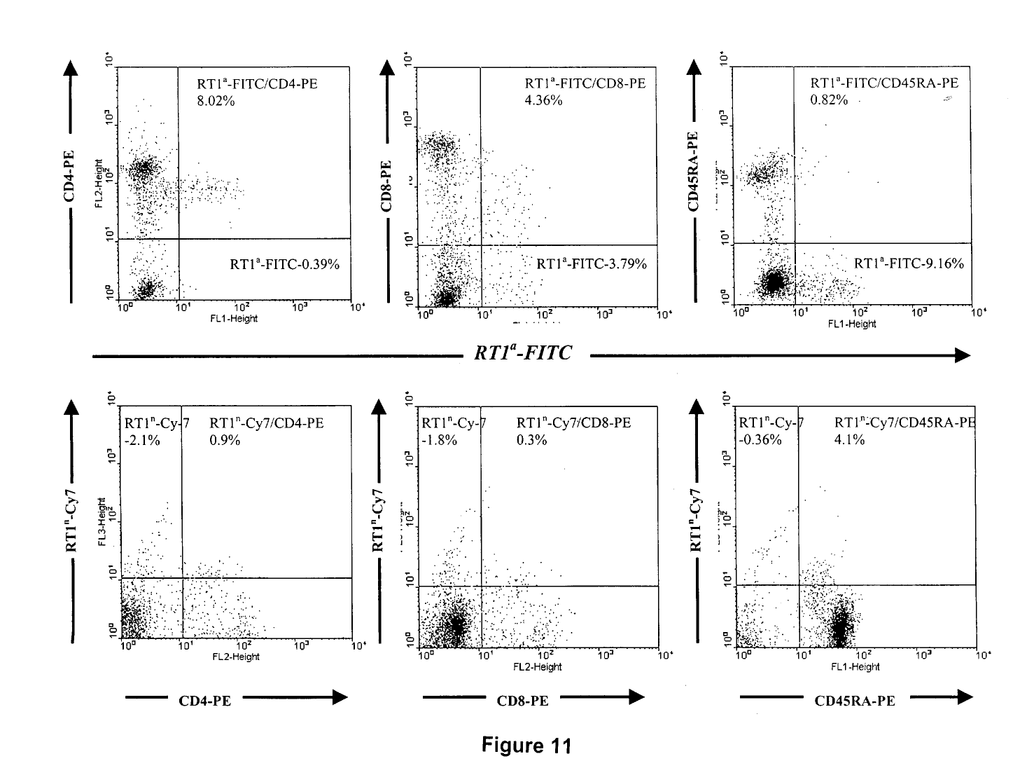

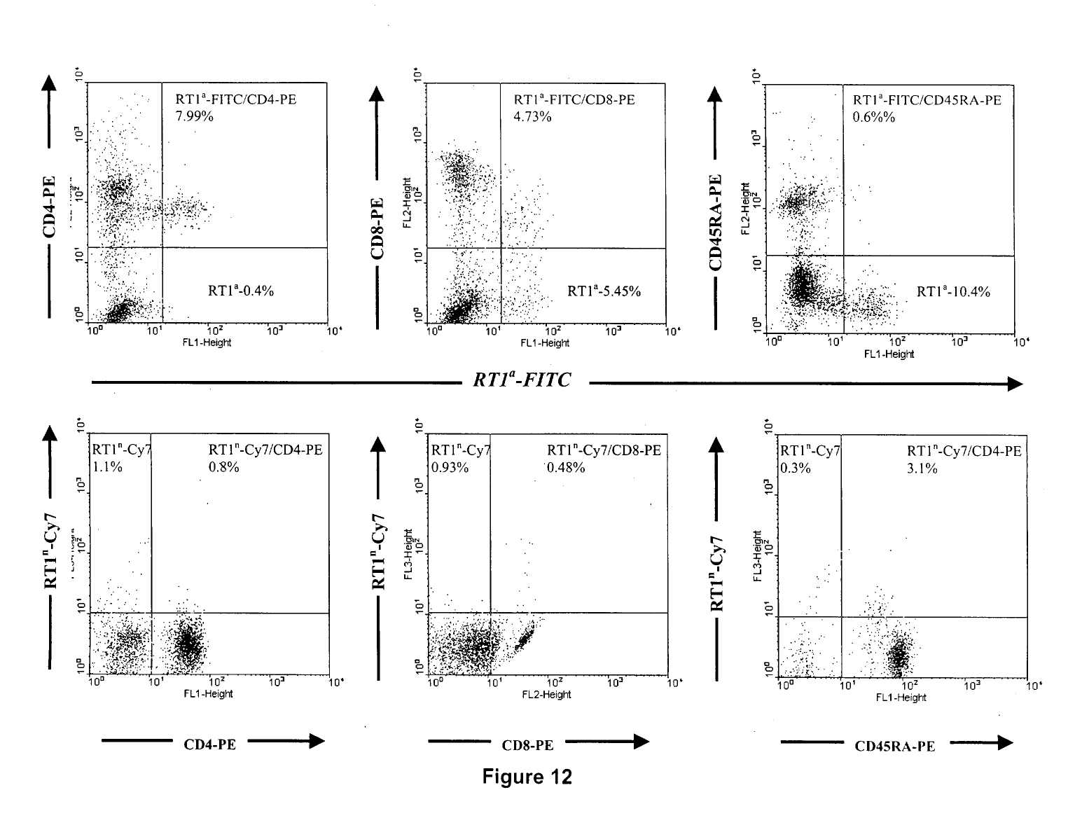

Determination of the Donor Specific Trimerism. Flow cytometry analysis was performed on PBMC of the LEW recipients at day 21 (FIG. 11) and day 35 (FIG. 12) after transplantation of the VSBAS transplants from the BN and ACI donors.

The dot-plot results of the lymphoid cell subpopulations obtained from quadrangle analytical gates demonstrated the presence of double positive RT-1^{a-FITC}/CD4^{-PE}, RT-1^{a-FITC/CD}8^{-PE} and RT-1^{a-FITC}/CD45RA^{-PE} (ACI/LEW) at a level of 8.02%, 4.36% and 0.82%, respectively, of PBMC on day 21 (FIG. 11, top horizontal row); and also the presence of double positive RT-1^{n-Cy7}/CD4^{-PE}, RT-1^{n-Cy7}/CD8^{-PE} and RT-1^{n-Cy7}/CD45RA^{-PE} (BN/LEW) at a level of 0.9%, 0.3% and 4.1%, respectively (FIG. 11, bottom horizontal row). The expression of RT-1^{a} (RT-1^{a-FITC}) antigen and RT-1ⁿ (RT-1^{n-Cy7}) antigen (FIG. 11, top horizontal row and bottom horizontal row, respectively) on non-CD4, non-CD8, and non-CD45RA positive T cell populations suggests the existence of donor/recipient trimeric residents not evaluated with the PE-conjugated mAb against surface specific antigen.

The dot plot results obtained from the peripheral blood of LEW recipients on day 35 are illustrated in FIG. 12. The results demonstrated the presence of double positive RT-1^{a-FITC}/CD4^{-PE}, RT-1^{a-FITC}/CD8^{-PE} and RT-1^{a-FITC}/CD45RA^{-PE} (ACI/LEW) at a level of 7.99%, 4.73% and 0.6%, respectively, of PBMC on day 35 (FIG. 12, top horizontal row); and also the presence of double positive RT-1^{n-Cu7}/CD4^{-PE}, RT-1^{n-Cy7}/CD8^{-PE} and RT-1^{n-Cy7}/CD45RA^{-PE} (BN/LEW) at a level of 0.8%, 0.48% and 3.1%, respectively (FIG. 12, bottom horizontal row).

These results show that the trimerism obtained in these LEW recipients was stable and correlated with the maintenance of tolerance to both VSBA fully allogeneic allografts.

In contrast, control LEW recipients receiving vascularized skin flaps without vascularized bone transplants showed a transient chimerism (less than 1% to 1%) that declined over time leading to allograft rejection within 40 days from the time of transplantation (data not shown).

Therefore, this example again illustrates the tolerance-inducing and chimerism/trimerism inducing properties of the vascularized bone component of the transplant. It has further been demonstrated that donor-specific cells can be produced in the recipient and can co-exist without rejection in the recipient. No recipient preconditioning is required.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

I claim:

1. A method for inducing donor-specific tolerance and/or mixed donor-recipient chimerism in an allograft transplant recipient, comprising the steps of:
(a) administering to a recipient of an allograft a therapeutically effective amount of an immunosuppressive agent that depletes T cells;
(b) administering to the recipient of the allograft a therapeutically effective amount of anti-αβ T cell receptor antibodies;
(c) implanting an allograft from an allograft donor into the recipient; and
(d) implanting a therapeutically effective amount of bone marrow cells from the allograft donor into the allograft recipient.

2. The method of claim 1, wherein the steps of administering the immunosuppressive agent and the anti-αβ T cell receptor antibodies, and implanting of the donor bone marrow cells, results in induction of hematopoietic mixed donor-recipient chimerism in the recipient.

3. The method of claim 1, wherein the therapeutically effective amount of allograft donor bone marrow cells is an amount sufficient to induce the production of mixed donor-recipient chimerism in the recipient.

4. The method of claim 3, wherein the mixed donor-recipient chimerism in the recipient comprises a level of mixed donor and recipient cells of about 5% to about 50% of circulating peripheral blood mononuclear cells.

5. The method of claim 4, wherein the level of mixed donor and recipient cells is about 10% to about 40% of circulating peripheral blood mononuclear cells.

6. The method of claim 5, wherein the level of mixed donor and recipient cells is about 20% to about 30% of circulating peripheral blood mononuclear cells.

7. The method of claim 1, wherein the therapeutically effective amount of the allograft donor bone marrow cells is an amount sufficient to maintain long-term recipient tolerance of the allograft without readministration of the immunosuppressive agent and the anti-αβ T cell receptor antibodies.

8. The method of claim 1, wherein the step of implanting bone marrow cells comprises the substeps of implanting and vascularizing an allograft donor bone including the bone marrow into the recipient.

9. The method of claim 8, wherein the allograft donor bone further includes an attached portion of donor skin, and wherein the substeps of implanting and vascularizing the donor bone into the recipient further includes the substeps of implanting and vascularizing the attached portion of donor skin.

10. The method of claim 1, wherein the step of implanting bone marrow cells comprises the substeps of obtaining crude bone marrow from a bone of the allograft donor, and implanting the donor crude bone marrow into the allograft recipient.

11. The method of claim 10, wherein the substep of implanting the allograft donor crude bone marrow further comprises the substeps of removing a quantity of crude bone marrow from a recipient bone marrow compartment to provide a space in the compartment, and directly implanting the allograft donor crude bone marrow into the space.

12. The method of claim 1, wherein the step of implanting bone marrow cells comprises the substeps of obtaining a bone marrow nucleated cell suspension from a bone of the allograft donor, and implanting the bone marrow nucleated cell suspension into the recipient.

13. The method of claim 12, wherein the bone marrow cell suspension is implanted by direct intraosseous injection of the suspension into a recipient bone.

14. The method of claim 12, wherein the bone marrow nucleated cell suspension is implanted by intravenous injection into the recipient.

15. The method of claim 1, wherein the step of implanting bone marrow cells comprises the substeps of obtaining a bone marrow cell suspension from a bone of the allograft donor; isolating a nucleated subpopulation of the bone marrow cells, and implanting the nucleated subpopulation of the bone marrow cells into the recipient.

16. The method of claim 15, wherein the subpopulation of the bone marrow cells is implanted by direct intraosseous injection of the suspension into a recipient bone.

17. The method of claim 15, wherein the subpopulation of the bone marrow cells is implanted by intravenous injection of the suspension into the recipient.

18. The method of claim 15, wherein the subpopulation of the isolated bone marrow cells includes tolerance inducing cells.

19. The method of claim 15, wherein the subpopulation of the isolated bone marrow cells includes CD4⁺ cells.

20. The method of claim 15, wherein the subpopulation of the isolated bone marrow cells includes CD8⁺ cells.

21. The method of claim 15, wherein the subpopulation of the isolated bone marrow cells includes hematopoietic stem cells.

22. The method of claim 15, wherein the subpopulation of the isolated bone marrow cells includes hematopoietic progenitor cells.

23. The method of claim 1, wherein the immunosuppressive agent and the anti-αβ T cell receptor antibodies are administered in an amount, at a frequency, and for a duration of time sufficient to eliminate about 50% to about 99.9% of recipient T cells circulating in peripheral blood of the recipient.

24. The method of claim 1, wherein the immunosuppressive agent comprises an IL-2 production inhibitor.

25. The method of claim 1, wherein the immunosuppressive agent comprises a calcineurin inhibitor.

26. The method of claim 1, wherein the immunosuppressive agent is selected from the group consisting of cyclosporine A, FK-506, rapamycin, and combinations thereof.

27. The method of claim 1, wherein the allograft is selected from the group consisting of composite tissue, solid organs, glands, glandular cells, skin, hematopoietic tissue, lymphoid tissue, tendons, ligaments, muscles, nerve tissue, vascular tissue and combinations thereof.

28. The method of claim 1, wherein the step of implanting the allograft does not include recipient preconditioning.

29. The method of claim 1, wherein the allograft recipient is a human.

30. The method of claim 29, wherein the anti-αβ TCR receptor antibodies are human antibodies to human αβ TCR⁺ T cells.

31. The method of claim 29, wherein the anti-αβ TCR receptor antibodies are humanized monoclonal antibodies to human αβ TCR⁺ T cells.

32. The method of claim 29, wherein the anti-αβ TCR receptor antibodies are human monoclonal antibodies to human αβ TCR⁺ T cells.

33. The method of claim 1, wherein the anti-αβ TCR receptor antibodies are monoclonal antibodies.

34. A method for inducing donor-specific tolerance and/or mixed donor-recipient trimerism in an allograft transplant recipient, comprising the steps of:
(a) administering to a recipient of an allograft a therapeutically effective amount of an immunosuppressive agent that depletes T cells;
(b) administering to the recipient of the allograft a therapeutically effective amount of anti-αβ T cell receptor antibodies;
(c) implanting a first allograft from a first allogeneic allograft donor into the recipient;
(d) implanting a therapeutically effective amount of bone marrow cells from the first allograft donor into the recipient.
(e) implanting a second allograft from a second allogeneic allograft donor into the recipient; and
(f) implanting a therapeutically effective amount of bone marrow cells from the second allograft donor into the allograft recipient,
wherein a state of trimerism is induced in the recipient.

35. The method of claim 34, wherein the first and second allogeneic allograft donors are independently selected from semi-allogeneic donors; fully allogeneic donors; and
combinations thereof, with respect to the recipient.

36. The method of claim 34, wherein the state of trimerism in the recipient comprises a level of mixed donor and recipient cells of about 0.1% to about 15% of circulating peripheral blood mononuclear cells.

37. The method of claim 34, wherein the state of trimerism in the recipient comprises a level of mixed donor and recipient cells of about 1% to about 10% of circulating peripheral blood mononuclear cells.

38. A method for monitoring allograft rejection in an allograft transplant recipient, comprising the steps of:
(a) administering to a recipient of an allograft a therapeutically effective amount of an immunosuppressive agent that depletes T cells;
(b) administering to the recipient of the allograft a therapeutically effective amount of anti-αβ T cell receptor antibodies;
(c) implanting an allograft from a donor into the recipient;
(d) implanting a therapeutically effective amount of bone marrow cells from the allograft donor into the recipient; and
(e) implanting an additional allograft from the donor into the recipient for monitoring allograft rejection in the recipient.

39. The method of claim 38, wherein the additional allograft is vascularized in the recipient.

40. The method of claim 39, wherein the additional allograft is skin.

41. The method of claim 38, wherein the additional allograft is unvascularized in the recipient.

42. The method of claim 41, wherein the additional allograft is skin.

43. The method of claim 38, wherein the allograft is selected from the group consisting of composite tissue, solid organs, glands, glandular cells, skin, hematopoietic tissue, lymphoid tissue, tendons, ligaments, muscles, nerve tissue, vascular tissue and combinations thereof.

44. A system for inducing donor-specific tolerance and/or mixed donor-recipient chimerism in an allograft transplant recipient, comprising:
(a) a combination of pharmaceutical compositions for depletion of T cells in a recipient of an allograft from a donor, comprising an effective amount of a pharmaceutical composition that comprises an immunosuppressive T cell-depleting agent, and an effective amount of a pharmaceutical composition that comprises anti-αβ TCR⁺ T cell receptor antibodies, wherein administration of the combination to the recipient results in elimination of about 50% to about 99.9% of T cells circulating in peripheral blood of the recipient;
(b) a delivery system for implanting a therapeutically effective amount of bone marrow cells from the allograft donor into the allograft recipient, wherein the delivery system is selected from the group consisting of an implantable vascularizable bone from the allograft donor including the donor bone marrow; implantable crude donor bone marrow; an implantable donor bone marrow nucleated cell suspension; an implantable isolated subpopulation of donor bone marrow cells; and combinations thereof.

45. The system of claim 44 wherein the implantable isolated subpopulation of bone marrow cells is selected from the group consisting of tolerance inducing cells, hematopoietic stem cells, hematopoietic progenitor cells, CD4⁺ cells, CD8⁺ cells, and combinations thereof.

46. The system of claim 44 wherein the effective amount of the combination of pharmaceutical compositions is sufficient to eliminate about 75% to about 95% of circulating T cells.

47. The system of claim 46 wherein the effective amount of the combination is sufficient to eliminate about 80% to about 90% of circulating T cells.

48. The system of claim 44 wherein the immunosuppressive agent comprises an IL-2 production inhibitor.

49. The system of claim 44, wherein the immunosuppressive agent comprises a calcineurin inhibitor.

50. The system of claim 44, wherein the immunosuppressive agent is selected from the group consisting of cyclosporine A, FK-506, rapamycin, and combinations thereof.

51. The system of claim 50, wherein the immunosuppressive agent comprises cyclosporine A.

52. The system of claim 44, wherein the recipient is a human.

53. The system of claim 52, wherein the anti-αβ TCR receptor antibodies are human antibodies to human αβ TCR⁺ T cells.

54. The system of claim 52, wherein the anti-αβ TCR receptor antibodies are humanized monoclonal antibodies to human αβ TCR⁺ T cells.

55. The system of claim 52, wherein the anti-αβ TCR receptor antibodies are human monoclonal antibodies to human αβ TCR⁺ T cells.

56. The system of claim 44, wherein the anti-αβ TCR receptor antibodies are monoclonal antibodies.

57. A method for maintaining a level of mixed donor-recipient chimerism in an allograft transplant recipient, comprising the steps of:
(a) inducing donor-specific tolerance and/or mixed donor-recipient chimerism in an allograft recipient by (i) administering to a recipient of an allograft a therapeutically effective amount of an immunosuppressive agent that depletes T cells; (ii) administering to the recipient of the allograft a therapeutically effective amount of anti-αβ T cell receptor antibodies; (iii) implanting an allograft from an allograft donor into the recipient; and (iv) implanting a therapeutically effective amount of bone marrow cells from the allograft donor into the allograft recipient;
(b) determining an optimal level of chimeric cells in the recipient not undergoing rejection of the allograft;
(c) harvesting chimeric cells from the recipient when the optimal level of chimeric cells is achieved;
(d) reconstituting the recipient with the harvested chimeric cells when the level of chimeric cells in the recipient falls below a minimum level of chimeric cells; and
(e) optionally readministering an effective amount of the immunosuppressive agent and/or the anti-αβ T cell receptor antibodies.

58. The method of claim 57, wherein the step of reconstituting the recipient comprises administering the chimeric cells by a method selected from the group consisting of direct intraosseous injection into a recipient bone marrow cavity; direct intraosseous injection into a bone marrow cavity of an implanted donor bone allograft; intravenous injection into the recipient; and combinations thereof.

59. The method of claim 57, wherein the minimum level of chimeric cells is about 5% to about 20% of circulating peripheral blood mononuclear cells.

60. The method of claim 57, wherein the optimal level of chimeric cells is about 10% to about 50% of circulating peripheral blood mononuclear cells.

61. The method of claim 60, wherein the optimal level of chimeric cells is about 20% to about 30% of circulating peripheral blood mononuclear cells.

62. The method of claim 57, wherein the harvesting step comprises isolating chimeric cells from the peripheral blood of the recipient.

63. The method of claim 57, wherein the harvesting step includes the substeps of isolating peripheral blood mononuclear cells from the recipient and, optionally, isolating chimeric cells from the isolated peripheral blood mononuclear cells.

64. The method of claim 57, wherein the harvesting step includes the substep of propagating the chimeric cells in culture prior to the reconstituting step.

65. The method of claim 57, wherein the harvesting step includes the substep of storing the chimeric cells prior to the reconstituting step.

66. The method of claim 65, wherein the substep of storing the chimeric cells comprises storing the cells at −196° C. in liquid nitrogen.

67. The method of claim 57, wherein the reconstituting step includes the step of obtaining a portion of the harvested chimeric cells and reconstituting the recipient with the portion.
